(19) United States
(12) Patent Application Publication
(10) Pub. No.: US 2015/0031572 A1
(43) Pub. Date: Jan. 29, 2015

(54) MEANS AND METHODS FOR ASSESSING NEURONAL TOXICITY

(71) Applicant: BASF SE, Ludwigshafen (DE)

(72) Inventors: Tilmann B. Walk, Kleinmachnow (DE); Bennard van Ravenzwaay, Altrip (DE); Werner Mellert, Hassloch (DE); Eric Fabian, Speyer (DE); Volker Strauss, Bad Durkheim (DE); Hennicke Kamp, Bischheim (DE); Jan C. Wiemer, Berlin (DE); Ralf Looser, Berlin (DE); Michael Manfred Herold, Berlin (DE); Alexandre Prokoudine, Berlin (DE)

(73) Assignee: BASF SE, Ludwigshafen (DE)

(21) Appl. No.: 14/377,603

(22) PCT Filed: Feb. 15, 2013
(86) PCT No.: PCT/IB2013/051230

§ 371 Date: Aug. 8, 2014

Related U.S. Application Data
(60) Provisional application No. 61/598,975, filed on Feb. 15, 2012.

(30) Foreign Application Priority Data
Feb. 15, 2012 (EP) 12155647.6

(51) Int. Cl. G01N 33/50; G01N 33/68; G01N 33/94; G01N 33/92

(52) U.S. Cl. CPC G01N 33/5014; G01N 33/5088; G01N 33/92; G01N 33/6848; G01N 33/942; G01N 2570/00; G01N 2405/04; G01N 2405/08; G01N 2405/02
USPC 506/9; 506/10; 435/7.92; 435/7.94; 436/71; 436/90; 436/96; 435/287.2; 422/82.01; 506/39

(57) ABSTRACT

The present invention pertains to the field of diagnostics for neuronal toxicity and toxicological assessments for risk stratification of chemical compounds. Specifically, it relates to a method for diagnosing neuronal toxicity. It also relates to a method for determining whether a compound is capable of inducing such neuronal toxicity in a subject and to a method of identifying a drug for treating neuronal toxicity. Furthermore, the present invention relates to a device and a kit for diagnosing neuronal toxicity.

The present invention pertains to the field of diagnostics for neuronal toxicity and toxicological assessments for risk stratification of chemical compounds. Specifically, it relates to a method for diagnosing neuronal toxicity. It also relates to a method for determining whether a compound is capable of inducing such neuronal toxicity in a subject and to a method of identifying a drug for treating neuronal toxicity. Furthermore, the present invention relates to a device and a kit for diagnosing neuronal toxicity.

The central nervous system (CNS) is the part of the nervous system that integrates the information that it receives from, and coordinates the activity of all parts of the bodies. The nervous system of all mammals has the same basic plan and physiological mechanisms, although some minor species variations do occur. The distribution of nervous tissue is not uniform throughout the body. In particular, there is a concentration along the midline axis within the bones of the cranium and the vertebral canal. These concentrations form the brain and spinal cord, respectively, and known as the central nervous system. On gross examination of the nervous system, areas of grey and white matter are visible: grey matter mainly consists of cell bodies, while white matter is mainly composed of myelinated nerve fibres. The central nervous system is protected by layers of connective tissue, the meninges (the outermost layer, the dura mater, the innermost layer, the pia mater) and extensions surround blood vessels as they enter and leave the central nervous system, producing perivascular spaces, the Virchow-Robin spaces, continuous with the subarachnoid space. Embryologically the neural tube undergoes exuberant differential growth to form the brain, while the remainder becomes the spinal cord. The brain consists of the forebrain (prosencephalon), the midbrain (mesencephalon) and the hindbrain (rhombencephalon).

Normal functioning of the nervous system is dependent on the innate ability of neurons to express excitability. Transmission of information is reliant on changes in the frequency and regularity of the generation of action potentials. Between neurons, and between neurons and receptor cells, there are synaptic junctions in which transmission occurs by the release of neurotransmitter. Some transmitters are excitatory others are inhibitory. This allows fine control over neuronal activity.

The brain may be divided also on a functional basis into various pathways containing cell groups and their axonal connections which utilize the same neurotransmitter. Five major pathways are recognized: (1) Cholinergic pathways of the basal part of the forebrain are concerned with memory, behaviour and mood, and those of the reticular formation of the brainstem influence the state of arousal and control sleep processes. (2) Noradrenergic pathways are restricted to the pontine and medullary regions but have connections with many regions of the central nervous system, including the limbic system. (3) Adrenergic neurons are confined to the caudal part of the hindbrain and the pathways are poorly understood. (4) Serotonergic pathways are found throughout the brainstem and regulate cardiovascular function, particularly cerebral blood flow, and control sleep processes. (5) Dopaminergic pathways are located in the mesencephalon and the hypothalamus.

A number of endogenous compounds function as neurotransmitters in the central nervous system. These include the substances, norepinephrine (NE), dopamine (DA), 5-hydroxytryptamine (serotonin, 5HT), acetylcholine (ACh), y-aminobutyric acid (GABA), phenylethylamine, histamine, glycine, glutamic acid, aspartic acid, taurine, and a number of peptides such as substance P, and the enkephalins. Neurotransmitters are stored in nerve terminals and are believed to be released from these terminals following stimulation of the nerve. A very complex machinery is known to exist which is responsible for the synthesis of these transmitter agents, their storage within the nerve terminals, and their release upon demand of the organism.

Dopamine is a catecholamine neurotransmitter present in a wide variety of animals. In the brain, this substituted phenethylamine activates the five known types of dopamine receptors D1, D2, D3, D4, and D5 and their variants. Dopamine is produced in several areas of the brain and is also a neurohormone released by the hypothalamus. Dopamine has many functions in the brain, including important roles in behavior and cognition, voluntary movement, motivation, punishment and reward, inhibition of prolactin production, sleep, mood, attention, working memory, and learning.

Glutamate is the main excitatory neurotransmitter of the brain and its effects are mediated by several subtypes of receptors called excitatory amino acid receptors (EAARs) those that are ligand-gated directly to ion channels (ionotropic) and those that are coupled with G proteins. The entry of glutamate into the CNS is regulated at the blood-brain barrier. Glutamate receptors mediate most of the excitatory neurotransmission in the mammalian central nervous system (CNS). They also participate in plastic changes of synaptic transmission underlying long-term potentiation in memory and learning, and the formation of neural networks during development.

Serotonin (5-hydroxytryptamine, 5-HT) modulates neural activity and a wide range of neuropsychological processes. However, most serotonin is found outside the central nervous system (gastrointestinal tract, platelets), and all serotonin receptors are expressed outside as well as within the brain. Serotonin regulates numerous biological processes including cardiovascular, pulmonary, gastrointestinal (GI), and genitourinary systems as well as the central nervous system (CNS) and dysregulation has been implicated in the pathogenesis of many psychiatric and neurological disorders. Serotonin modulates virtually all human behavioral processes.

Norepinephrine or noradrenaline is synthesized from dopamine by dopamine β-hydroxylase. It is a catecholamine with multiple functions including hormone and a neurotransmitter in the central nervous system and sympathetic nervous system released from noradrenergic neurons. Norepinephrine affects parts of the brain, where attention and responses are controlled and underlies the fight-or-flight response, directly increasing heart rate/blood pressure, triggering the release of glucose from energy stores, and increasing blood flow to skeletal muscle and increases the brain's oxygen supply.

Nicotine (methylpyridylpyrrolidine) exerts its effect by binding to certain types of cholinergic receptors, the nicotinic receptors, at synapses in the central nervous system, the peripheral ganglia and the neuromuscular junctions, where it acts as an acetylcholine (AC) agonist. In small concentrations, nicotine increases the activity of these receptors. Nicotine also has effects on a variety of other neurotransmitters. Nicotine has a higher affinity for acetylcholine receptors in the brain than those in skeletal muscle, though at toxic doses it can induce contractions and respiratory paralysis.

Clinical effects include sweating, tachycardia, elevated blood pressure and constriction of cutaneous blood vessels. Nicotine in the appropriate area of the CNS has psychoactive actions. It directly stimulates the nicotine subset of CNS and peripheral AC receptors. Moderate poisoning produce symptoms of cholinergic excess including miosis, salivation, urination, defecation, emesis, and increased pulmonary secretions. In large-dose exposure a short live stimulation is followed rapidly by neuromuscular blockade related to persistent membrane depolarization. If death occurs, the most common mechanism is respiratory arrest due to peripheral neuromuscular blockade and cardiovascular collapse.

Exposure to a variety of drugs and toxins can induce an adverse change in the structure or function of the central nervous system and interpretation of morphological and functional CNS disturbances due to impaired neurotransmitter action in a toxicological setting may be quite complex and may involve both local as well as systemic manifestations of toxicity and/or pharmacologic response. In a general way, these changes can be classified as either quantitative or qualitative. A large number of naturally occurring toxins, as well as many synthetic molecules, cause neurotoxicity by interfering with the processes of chemical transmission between neurons, or between neurons and other cell types, notably skeletal muscle cells. This interference may block either the transmission of impulses or result in accentuation of the neurotransmission, depending on the mechanism of action and on the type of synapse affected. The effect may be transient or permanent. Compounds with similar actions usually have similar chemical and structural properties, and act only on one particular type of synapse. Mechanisms of action differ: some compounds act as agonists or antagonists to the chemical transmitter, while others act by preventing synthesis or release of the neurotransmitter, or by preventing its inactivation or resorption after release.

Due to the diversity of possible actions, the assessment of CNS toxicity with regards to effects on specific neurotransmitter is a rather complex process. The current methods usually comprise enhanced clinical observation, functional observation battery, motor activity, pathological and histopathological investigations as well as a biochemical analysis. However, the biomarkers are rather complex regulated and changes may sometimes occur even at rather progressed stages. Major drawbacks of the histopathological assessments are that they are invasive, and even when combined with the clinical pathology/hematology measurements that they are less reliable because they are in part based an the individual interpretations of toxicologist carrying out the investigations. (Berger 2009, The Expanded Biology of Serotonin, Annu. Rev. Med., 60, 355-366; Buckley P (1998) Chapter 9—The nervous system, in: Target organ pathology, a basic text, Turton J and Hooson J (eds) Taylor & Francis, London, United Kingdom, 1998; Mandella R C (2002) Chapter 8—Applied Neurotoxicology, in: CRC Handbook of Toxicology, 2nd edition, Derelanko M J and Hollinger M A (eds.) Taylor & Francis, London, United Kingdom, 2002; Moser V C, Aschner M, Richardson R J, Philbert M A (2008) Chapter 16. Toxic responses of the nervous system, 631-664, in: Casarett & Doull's Toxicology, The basic science of poisons, Klaassen C D (ed.), McGraw-Hill P, 7th revised edition, New York (2008); Ozawa 1998, Glutamate receptors in the mammalian central nervous system, Progress in Neurobiology, 54, 581-618; Schulze G E (2002) Chapter 7—Fundamental Neurotoxicology, in: CRC Handbook of Toxicology, 2nd edition, Derelanko M J and Hollinger M A (eds.) Taylor & Francis, London, United Kingdom, 2002)

Sensitive and specific methods for determining efficiently and reliably CNS toxicity with regards to effects on specific neurotransmitter and, in particular, the early onset thereof is not available but would, nevertheless, be highly appreciated. The importance of CNS (neurotransmitter-associated) toxicity may become apparent if one considers its wide range of mechanisms to maintain physiological and psychological homoeostasis and its consequences on all of the other organ systems of the body controlled by the nervous system. Thus damage to this “master” system can have far-reaching and even devastating effects. Moreover, chemical compounds which are used in any kind of industry in the European Community, e.g., will now need to comply with REACH (Registration, Evaluation and Authorisation of Chemicals). In other countries, similar toxicological risk assessments need to be done, e.g., the Material Safety Data Sheets (MSDS) in the US. It will be understood that the potential of a chemical compound to induce CNS toxicity with effects on specific neurotransmitter will be deemed as a high risk for the compound and, consequently, the compound will be available only for limited applications and when obeying high security standards.

The eye is also a structure of the CNS and can be effected by CNS toxicity as well. It is a complex organ but it has one function only, namely photosensory reception. The structural elements contributing to this function include (i) neurosensory retina; (ii) light-transmitting structures—cornea, lens, aqueous and vitreous humors; (iii) rigid structures—tough sclera and anterior cornea retaining the fluid contents; and (iv) the uveal tract—comprising the choroid and ciliary processes which provide oxygen and nutrition, and the iris which acts as a variable diaphragm regulating the light entering. The outer protective tissues of the eye are composed of the cornea, the conjunctiva, and the sclera. The middle vascular layer of the eye is composed of the iris, the ciliary body, and the choroid. A biconvex, avascular, colorless, and almost completely transparent structure suspended behind the iris by the zonula, which connects to the ciliary body. The zonula or suspensory ligament of the lens, is composed of numerous fibrils arising from the ciliary body and inserting into the equator of the lens. The aqueous is anterior to the lens and the vitreous is posterior to it. The lens is encapsulated by a semipermeable membrane, the lens capsule.

The cornea is unique because of its transparency. Corneal transparency is dependent on a special arrangement of cells and collagenous fibrils in an acid mucopolysaccharide detergence. Any toxin interfering with any one of these factors may result in corneal opacification. The cornea is composed of five distinct layers: (1) epithelium, (2) Bowman's membrane, (3) stroma, (4) Descemet's membrane and (5) endothelium.

There are many ophthalmologic procedures for evaluating the health of the eye. Procedures available range from fairly routine clinical screening evaluations to sophisticated techniques for much targeted purposes. A clinical evaluation of the eye addresses the adnexa and both, the anterior and posterior structures in the eye. The anterior structures or anterior segment include the cornea, iris, lens, and anterior chamber. The posterior structures, referred to as the ocular fundus, include the retina, retinal vasculature, choroid, and sclera.

The eye consists of a wide variety of tissue types and morphological structures with different biochemical processes which make it potentially susceptible to the toxic effects of many chemicals. With reference to toxicological conditions, the number of reported instances of altered structure or function of the eye due to toxic substances are exceeded only by the liver. Therefore a sound knowledge of the pathological processes occurring after toxicological insult is extremely important. Unlike some organ systems of the body, the biochemical and cellular mechanisms for many known toxic effects in the eye are poorly understood.

Corneal injury from systemic administration may take the form of keratitis, edema or opacification from the deposition of endogenous or exogenous substances. Reported examples of keratitis in this type of injury are few, although the administration of tyrosine in a low protein diet is reported to induce keratitis in rats.

Tyrosine has a special role by virtue of the phenol functionality aside from being a proteogenic amino acid. It occurs in proteins that are part of signal transduction processes. It functions as a receiver of phosphate groups that are transferred by way of protein kinases (so-called receptor tyrosine kinases). Phosphorylation of the hydroxyl group changes the activity of the target protein.

Interest in the effects of excessive intakes of tyrosine on experimental animals has as its basis the need to understand the changes in tissue and in metabolism of tyrosine in individuals with the inherited disorder of tyrosine metabolism, tyrosinemia II. Tyrosinemia II is inherited as an autosomal recessive trait and involves a complete lack of the liver enzyme tyrosine aminotransferase associated with tyrosinemia, tyrosinuria, and increased urinary excretion of tyrosine metabolites. Individuals with this disease have corneal lesions and palm and sole erosions in the early months of life. Major progress in understanding the disease has come from observation of laboratory rats fed low-protein diets containing high levels of tyrosine and from studies of mink that have an inherited disorder of tyrosine metabolism.

High concentration of plasma and tissue tyrosine causes eye and paw lesions. Within 24 hours of the initiation of feeding diets containing 5% of tyrosine to laboratory rats, alterations appear in the cornea, which are described as dots that progress into “snow flake” opacity. The cellular architecture becomes distorted, the cornea thickens and becomes opaque. The positive identification of crystals within the cells of the cornea of rats fed low-protein diets containing tyrosine has been made. Factors that prevent tyrosine toxicity in rats also prevent crystal formation and corneal lesions.

In addition, rats exposed to sulcotrione revealed corneal lesions that varied in severity from partial or hazy opacity to complete opacity, with edema and neovascularization evident in the more extensive lesions. Tyrosine concentration could be as much as 10 times the normal value after 24 hours when rats were given the sulcotrione analogue in a single dose. It also caused cornea damage. Corneal opacity in individual rats was also found, which might be related to the accumulation of excessive tyrosine in the anterior chamber of eye.

Interpretation of eye changes with regards to tyrosine in a toxicological setting may be quite complex as the heterogeneous nature of the eye and its composition of different tissue types renders it particularly susceptible to toxicity. This spectrum of anatomical and functional features provides a wide variety of pathological responses which are important in toxicology. The eye is notorious for demonstrating marked species variation in its response to toxic chemicals. This, together with the generally poor understanding of mechanisms with respect to ocular toxicity, renders the extrapolation of the effects of toxic chemicals in laboratory animals to man extremely difficult. To make broad generalizations on this subject is fraught with hazard, except to regard experimental oculotoxicity testing as being of value only to identify the potential of a chemical to cause ocular damage in man.

Due to the diversity of possible actions, the assessment of eye toxicity with regards to tyrosine is a rather complex process. The current methods usually comprise naked eye examination with suitable illumination. For more detailed available, e.g. direct and indirect ophthalmoscopes, hand-held or table-mounted slit lamps and fundic cameras. Careful attention is required in fixation and processing of eyes for histology. Since the eye is composed of a variety of tissue types, the ideal fixative for a particular purpose is generally the best compromise available. Careful searching for focal lesions is important and a combination of ophthalmoscopy and histology is ideal. Major drawbacks of the histopathological assessments are that they are invasive, and even when combined with the clinical pathology/hematology measurements that they are less reliable because they are in part based an the individual interpretations of toxicologist carrying out the investigations. (Benevenga N J, Steele (1984) Adverse effects of excessive consumption of amino acids, Ann. Rev. Nutr., 4, 157-181; Fox D A, Boyes W K (2008) Chapter 17, Toxic responses of the ocular and visual system, 665-698, in: Casarett & Doull's Toxicology, The basic science of poisons, Klaassen C D (ed.), McGraw-Hill P, 7th revised edition, New York (2008); Goldsmith L A, Reed J (1976) Tyrosine-induced eye and skin lesions, a treatable genetic disease, JAMA, 236, 382-384; McCaa C S (1982) The eye and visual nervous system: Anatomy, physiology and toxicology, Environ. Health Perspect., 44, 1-8; Robinson M (1998) Chapter 15, Organs of Special Sense 1: The Eye, in: Target organ pathology, a basic text, Turton J and Hooson J (eds) Taylor & Francis, London, United Kingdom, 1998)

Sensitive and specific methods for determining efficiently and reliably eye toxicity with regards to tyrosine and, in particular, the early onset thereof are not available but would, nevertheless, be highly appreciated. The importance of eye toxicity may become apparent if one considers its consequences on vision and wellbeing. Moreover, chemical compounds which are used in any kind of industry in the European Community, e.g., will now need to comply with REACH (Registration, Evaluation and Authorisation of Chemicals). In other countries, similar toxicological risk assessments need to be done, e.g., the Material Safety Data Sheets (MSDS) in the US. It will be understood that the potential of a chemical compound to induce eye toxicity will be deemed as a high risk for the compound and, consequently, the compound will be available only for limited applications and when obeying high security standards.

Sensitive and specific methods for assessing the toxicological properties of a chemical compound and, in particular, neuronal toxicity, in an efficient and reliable manner are not yet available but would, nevertheless, be highly appreciated.

Thus, the technical problem underlying the present invention could be seen as the provision of means and methods for complying with the aforementioned needs. The technical problem is solved by the embodiments characterized in the claims and described herein below.

Accordingly, the present invention relates to a method for diagnosing neuronal toxicity comprising:

(a) determining the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9a, 9b, 9c, 9d, 12a or 12b in a test sample of a subject suspected to suffer from neuronal toxicity, and
(b) comparing the amounts determined in step (a) to a reference, whereby neuronal toxicity is to be diagnosed.

In a particular embodiment of the method of the invention, a method is provided for diagnosing neuronal toxicity comprising:

(a) selecting a male or female subject suspected to suffer from neuronal toxicity;
(b) obtaining a test sample from said selected subject;
(c) pre-treating said sample in preparation for analysis;
(d) determining the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9a, 9b, 9c, 9d, 12a or 12b in said test sample, and
(e) comparing the amounts determined in step (d) to a reference; and
(f) based on the comparison of step (e), diagnose neuronal toxicity by monitoring, confirmation or classification of the neuronal toxicity or its symptoms.

In a preferred embodiment of the aforementioned method said subject has been brought into contact with a compound suspected to be capable of inducing neuronal toxicity.

The present invention also relates to a method of determining whether a compound is capable of inducing neuronal toxicity in a subject comprising:

(a) determining in a sample of a subject which has been brought into contact with a compound suspected to be capable of inducing neuronal toxicity the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9a, 9b, 9c, 9d, 12a or 12b; and
(b) comparing the amounts determined in step (a) to a reference, whereby the capability of the compound to induce neuronal toxicity is determined.

In a particular embodiment of the method of the invention, a method is provided for determining whether a compound is capable of inducing neuronal toxicity in a subject comprising:

(a1) (i) selecting a male or female subject;
(ii) bringing said subject into contact with a compound suspected to be capable of inducing neuronal toxicity, or
(a2) selecting a male or female subject brought into contact with a compound capable of inducing neuronal toxicity;
(b) obtaining a test sample from said selected subject;
(c) pre-treating said sample in preparation for analysis;
(d) determining the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9a, 9b, 9c, 9d, 12a or 12b in said test sample, and
(e) comparing the amounts determined in step (d) to a reference; and
(f) based on the comparison of step (e), identifying whether the compound is capable of inducing neuronal toxicity, or not.

In a preferred embodiment of the aforementioned method said compound is at least one compound selected from the group consisting of: 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, and Thallium(I) acetate.

In another preferred embodiment of the methods of the present invention said reference is derived from (i) a subject or group of subjects which suffers from neuronal toxicity or (ii) a subject or group of subjects which has been brought into contact with at least one compound selected from the group consisting of: 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, and Thallium(I) acetate. In a more preferred embodiment of said method essentially identical amounts for the biomarkers in the test sample and the reference are indicative for neuronal toxicity.

In another preferred embodiment of the methods of the present invention said reference is derived from (i) a subject or group of subjects known to not suffer from neuronal toxicity or (ii) a subject or group of subjects which has not been brought into contact with at least one compound selected from the group consisting of: 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, and Thallium(I) acetate. In a more preferred embodiment of said methods amounts for the biomarkers which differ in the test sample in comparison to the reference are indicative for neuronal toxicity.

In yet another embodiment of the methods of the present invention said reference is a calculated reference for the biomarkers for a population of subjects. In a more preferred embodiment of said methods amounts for the biomarkers which differ in the test sample in comparison to the reference are indicative for neuronal toxicity.

The present invention also contemplates a method of identifying a substance for treating neuronal toxicity comprising the steps of:

(a) determining in a sample of a subject suffering from neuronal toxicity which has been brought into contact with a candidate substance suspected to be capable of treating neuronal toxicity the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9a, 9b, 9c, 9d, 12a or 12b; and
(b) comparing the amounts determined in step (a) to a reference, whereby a substance capable of treating neuronal toxicity is to be identified.

In a particular embodiment of the method of the invention, a method is provided for identifying a substance for treating neuronal toxicity comprising:

(a1) (i) selecting a male or female subject;
(ii) bringing said subject into contact with a compound suspected to be capable of inducing neuronal toxicity such that neuronal toxicity is elicited, or
(a2) selecting a male or female suffering from neuronal toxicity;
(b) obtaining a test sample from said selected subject;
(c) pre-treating said sample in preparation for analysis;
(d) determining the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9a, 9b, 9c, 9d, 12a or 12b in said test sample, and
(e) comparing the amounts determined in step (d) to a reference; and
(f) based on the comparison of step (e), identifying and selecting the substance for treating neuronal toxicity.

In a preferred embodiment of the aforementioned method said reference is derived from (i) a subject or group of subjects which suffers from neuronal toxicity or (ii) a subject or group of subjects which has been brought into contact with at least one compound selected from the group consisting of: 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, and Toxaphene, Glipizide, Lead acetate trihydrate, and Thallium(I) acetate. In a more preferred embodiment of said method amounts for the biomarkers which differ in the test sample and the reference are indicative for a substance capable of treating neuronal toxicity.

In another preferred embodiment of the aforementioned method said reference is derived from (i) a subject or group of subjects known to not suffer from neuronal toxicity or (ii) a subject or group of subjects which has not been brought into contact with at least one compound selected from the group consisting of: 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Rispendone, Selegiline hydrochloride, Sertraline hydrochloride, Ziprasidone hydrochloride, Toxaphene, Glipizide, Lead acetate trihydrate, and Thallium(I) acetate. In a more preferred embodiment of the said methods essentially identical amounts for the biomarkers in the test sample and the reference are indicative for a substance capable of treating neuronal toxicity.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating neuronal toxicity.

The present invention also relates to the use of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9a, 9b, 9c, 9d, 12a or 12b or a detection agent for the said biomarker for diagnosing neuronal toxicity in a sample of a subject.

Moreover, the present invention relates to a device for diagnosing neuronal toxicity in a sample of a subject suspected to suffer therefrom comprising:

(a) an analyzing unit comprising a detection agent for at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9a, 9b, 9c, 9d, 12a or 12b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby neuronal toxicity is diagnosed.

In a preferred embodiment of the device of the invention said stored reference is a reference derived from a subject or a group of subjects known to suffer from neuronal toxicity or a subject or group of subjects which has been brought into contact with at least one compound selected from the group consisting of 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, and Thallium(I) acetate, and said data processor executes instructions for comparing the amount of the at least one biomarker determined by the analyzing unit to the stored reference, wherein an essentially identical amount of the at least one biomarker in the test sample in comparison to the reference is indicative for the presence of neuronal toxicity or wherein an amount of the at least one biomarker in the test sample which differs in comparison to the reference is indicative for the absence of neuronal toxicity.

In another preferred embodiment of the device of the invention said stored reference is a reference derived from a subject or a group of subjects known to not suffer from neuronal toxicity or a subject or group of subjects which has not been brought into contact with at least one compound selected from the group consisting of 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, and Thallium(I) acetate, and said data processor executes instructions for comparing the amount of the at least one biomarker determined by the analyzing unit to the stored reference, wherein an amount of the at least one biomarker in the test sample which differs in comparison to the reference is indicative for the presence of neuronal toxicity or wherein an essential identical amount of the at least one biomarker in the test sample in comparison to the reference is indicative for the absence of neuronal toxicity.

Further, the present invention relates to a kit for diagnosing neuronal toxicity comprising a detection agent for the at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9a, 9b, 9c, 9d, 12a or 12b and standards for the at least one biomarker the concentration of which is derived from a subject or a group of subjects known to suffer from neuronal toxicity or derived from a subject or a group of subjects known to not suffer from neuronal toxicity.

In particular, the present invention relates to a method for diagnosing CNS toxicity comprising:

(a) determining the amount of at least one biomarker selected from any one of Tables 1a, 1b, 1c, 1d, 2a, 2b, 3a, 3b, 4a, 4b, 5a, 5b, 6a, 6b, 6c, 6d, 7a, 7b, 8a or 8b in a test sample of a subject suspected to suffer from CNS toxicity, and
(b) comparing the amounts determined in step (a) to a reference, whereby CNS toxicity is to be diagnosed.

In a particular embodiment of the method of the invention, a method is provided for diagnosing CNS toxicity comprising:

(a) selecting a male or female subject suspected to suffer from CNS toxicity;
(b) obtaining a test sample from said selected subject;
(c) pre-treating said sample in preparation for analysis;
(d) determining the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a or 8b in said test sample, and
(e) comparing the amounts determined in step (d) to a reference; and
(f) based on the comparison of step (e), diagnose CNS toxicity by monitoring, confirmation or classification of the CNS toxicity or its symptoms.

In a preferred embodiment of the aforementioned method said subject has been brought into contact with a compound suspected to be capable of inducing CNS toxicity.

The present invention also relates to a method of determining whether a compound is capable of inducing CNS toxicity in a subject comprising:

(a) determining in a sample of a subject which has been brought into contact with a compound suspected to be capable of inducing CNS toxicity the amount of at least one biomarker selected from any one of Tables 1a, 1b, 1c, 1d, 2a, 2b, 3a, 3b, 4a, 4b, 5a, 5b, 6a, 6b, 6c, 6d, 7a, 7b, 8a or 8b; and
(b) comparing the amounts determined in step (a) to a reference, whereby the capability of the compound to induce CNS toxicity is determined.

In a particular embodiment of the method of the invention, a method is provided for determining whether a compound is capable of inducing CNS toxicity in a subject comprising:

(a1) (i) selecting a male or female subject;
(ii) bringing said subject into contact with a compound suspected to be capable of inducing CNS toxicity, or
(a2) selecting a male or female subject brought into contact with a compound capable of inducing CNS toxicity;
(b) obtaining a test sample from said selected subject;
(c) pre-treating said sample in preparation for analysis;
(d) determining the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a or 8b in said test sample, and
(e) comparing the amounts determined in step (d) to a reference; and
(f) based on the comparison of step (e), identifying whether the compound is capable of inducing CNS toxicity, or not.

In a preferred embodiment of the aforementioned method said compound is at least one compound selected from the group consisting of: 17-alpha-Ethynylestradiol, Apomorphine, Bromocriptine Mesylate, Cabergoline, Chlorpromazine, Citalopram hydrobromide, Dextroamphetamine sulfate, Escitalopram oxalate, Fluoxetine hydrochloride, Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, and Ziprasidone hydrochloride.

In another preferred embodiment of the methods of the present invention said reference is derived from (i) a subject or group of subjects which suffers from CNS toxicity or (ii) a subject or group of subjects which has been brought into contact with at least one compound selected from the group consisting of: 17-alpha-Ethynylestradiol, Apomorphine, Bromocriptine Mesylate, Cabergoline, Chlorpromazine, Citalopram hydrobromide, Dextroamphetamine sulfate, Escitalopram oxalate, Fluoxetine hydrochloride, Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, and Ziprasidone hydrochloride. In a more preferred embodiment of said method essentially identical amounts for the biomarkers in the test sample and the reference are indicative for CNS toxicity.

In another preferred embodiment of the methods of the present invention said reference is derived from (i) a subject or group of subjects known to not suffer from CNS toxicity or (ii) a subject or group of subjects which has not been brought into contact with at least one compound selected from the group consisting of: 17-alpha-Ethynylestradiol, Apomorphine, Bromocriptine Mesylate, Cabergoline, Chlorpromazine, Citalopram hydrobromide, Dextroamphetamine sulfate, Escitalopram oxalate, Fluoxetine hydrochloride, Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, and Ziprasidone hydrochloride. In a more preferred embodiment of said methods amounts for the biomarkers which differ in the test sample in comparison to the reference are indicative for CNS toxicity.

In yet another embodiment of the methods of the present invention said reference is a calculated reference for the biomarkers for a population of subjects. In a more preferred embodiment of said methods amounts for the biomarkers which differ in the test sample in comparison to the reference are indicative for CNS toxicity.

The present invention also contemplates a method of identifying a substance for treating CNS toxicity comprising the steps of:

(a) determining in a sample of a subject suffering from CNS toxicity which has been brought into contact with a candidate substance suspected to be capable of treating CNS toxicity the amount of at least one biomarker selected from any one of Tables 1a, 1b, 1c, 1d, 2a, 2b, 3a, 3b, 4a, 4b, 5a, 5b, 6a, 6b, 6c, 6d, 7a, 7b, 8a or 8b; and
(b) comparing the amounts determined in step (a) to a reference, whereby a substance capable of treating CNS toxicity is to be identified.

In a particular embodiment of the method of the invention, a method is provided for identifying a substance for treating CNS toxicity comprising:

(a1) (i) selecting a male or female subject;
(ii) bringing said subject into contact with a compound suspected to be capable of inducing CNS toxicity such that CNS toxicity is elicited, or
(a2) selecting a male or female suffering from CNS toxicity;
(b) obtaining a test sample from said selected subject;
(c) pre-treating said sample in preparation for analysis;
(d) determining the amount of at least one biomarker selected from any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a or 8b in said test sample, and
(e) comparing the amounts determined in step (d) to a reference; and
(f) based on the comparison of step (e), identifying and selecting the substance for treating CNS toxicity.

In a preferred embodiment of the aforementioned method said reference is derived from (i) a subject or group of subjects which suffers from CNS toxicity or (ii) a subject or group of subjects which has been brought into contact with at least one compound selected from the group consisting of: 17-alpha-Ethynylestradiol, Apomorphine, Bromocriptine Mesylate, Cabergoline, Chlorpromazine, Citalopram hydrobromide, Dextroamphetamine sulfate, Escitalopram oxalate, Fluoxetine hydrochloride, Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, and Ziprasidone hydrochloride. In a more preferred embodiment of said method amounts for the biomarkers which differ in the test sample and the reference are indicative for a substance capable of treating CNS toxicity.

In another preferred embodiment of the aforementioned method said reference is derived from (i) a subject or group of subjects known to not suffer from CNS toxicity or (ii) a subject or group of subjects which has not been brought into contact with at least one compound selected from the group consisting of: 17-alpha-Ethynylestradiol, Apomorphine, Bromocriptine Mesylate, Cabergoline, Chlorpromazine, Citalopram hydrobromide, Dextroamphetamine sulfate, Escitalopram oxalate, Fluoxetine hydrochloride, Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, and Ziprasidone hydrochloride. In a more preferred embodiment of the said methods essentially identical amounts for the biomarkers in the test sample and the reference are indicative for a substance capable of treating CNS toxicity.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating CNS toxicity.

The present invention also relates to the use of at least one biomarker selected from any one of Tables 1a, 1b, 1c, 1d, 2a, 2b, 3a, 3b, 4a, 4b, 5a, 5b, 6a, 6b, 6c, 6d, 7a, 7b, 8a or 8b or a detection agent for the said biomarker for diagnosing CNS toxicity in a sample of a subject.

Moreover, the present invention relates to a device for diagnosing CNS toxicity in a sample of a subject suspected to suffer therefrom comprising:

(a) an analyzing unit comprising a detection agent for at least one biomarker selected from any one of Tables 1a, 1b, 1c, 1d, 2a, 2b, 3a, 3b, 4a, 4b, 5a, 5b, 6a, 6b, 6c, 6d, 7a, 7b, 8a or 8b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby CNS toxicity is diagnosed.

In a preferred embodiment of the device of the invention said stored reference is a reference derived from a subject or a group of subjects known to suffer from CNS toxicity or a subject or group of subjects which has been brought into contact with at least one compound selected from the group consisting of 17-alpha-Ethynylestradiol, Apomorphine, Bromocriptine Mesylate, Cabergoline, Chlorpromazine, Citalopram hydrobromide, Dextroamphetamine sulfate, Escitalopram oxalate, Fluoxetine hydrochloride, Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, and Ziprasidone hydrochloride, and said data processor executes instructions for comparing the amount of the at least one biomarker determined by the analyzing unit to the stored reference, wherein an essentially identical amount of the at least one biomarker in the test sample in comparison to the reference is indicative for the presence of CNS toxicity or wherein an amount of the at least one biomarker in the test sample which differs in comparison to the reference is indicative for the absence of CNS toxicity.

In another preferred embodiment of the device of the invention said stored reference is a reference derived from a subject or a group of subjects known to not suffer from CNS toxicity or a subject or group of subjects which has not been brought into contact with at least one compound selected from the group consisting of 17-alpha-Ethynylestradiol, Apomorphine, Bromocriptine Mesylate, Cabergoline, Chlorpromazine, Citalopram hydrobromide, Dextroamphetamine sulfate, Escitalopram oxalate, Fluoxetine hydrochloride, Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, and Ziprasidone hydrochloride, and said data processor executes instructions for comparing the amount of the at least one biomarker determined by the analyzing unit to the stored reference, wherein an amount of the at least one biomarker in the test sample which differs in comparison to the reference is indicative for the presence of CNS toxicity or wherein an essential identical amount of the at least one biomarker in the test sample in comparison to the reference is indicative for the absence of CNS toxicity.

Further, the present invention relates to a kit for diagnosing CNS toxicity comprising a detection agent for the at least one biomarker selected from any one of Tables 1a, 1b, 1c, 1d, 2a, 2b, 3a, 3b, 4a, 4b, 5a, 5b, 6a, 6b, 6c, 6d, 7a, 7b, 8a, or 8b and standards for the at least one biomarker the concentration of which is derived from a subject or a group of subjects known to suffer from CNS toxicity or derived from a subject or a group of subjects known to not suffer from CNS toxicity.

In particular, the present invention relates to a method for diagnosing eye toxicity comprising:

(a) determining the amount of at least one biomarker selected from any one of Tables 9a, 9b, 9c, or 9d in a test sample of a subject suspected to suffer from eye toxicity, and
(b) comparing the amounts determined in step (a) to a reference, whereby eye toxicity is to be diagnosed.

In a particular embodiment of the method of the invention, a method is provided for diagnosing eye toxicity comprising:

(a) selecting a male or female subject suspected to suffer from eye toxicity;
(b) obtaining a test sample from said selected subject;
(c) pre-treating said sample in preparation for analysis;
(d) determining the amount of at least one biomarker selected from any one of Tables 9a, 9b, 9c or 9d in said test sample, and
(e) comparing the amounts determined in step (d) to a reference; and
(f) based on the comparison of step (e), diagnose eye toxicity by monitoring, confirmation or classification of the eye toxicity or its symptoms.

In a preferred embodiment of the aforementioned method said subject has been brought into contact with a compound suspected to be capable of inducing eye toxicity.

The present invention also relates to a method of determining whether a compound is capable of inducing eye toxicity in a subject comprising:

(a) determining in a sample of a subject which has been brought into contact with a compound suspected to be capable of inducing eye toxicity the amount of at least one biomarker selected from any one of Tables 9a, 9b, 9c, or 9d; and
(b) comparing the amounts determined in step (a) to a reference, whereby the capability of the compound to induce eye toxicity is determined.

In a particular embodiment of the method of the invention, a method is provided for determining whether a compound is capable of inducing eye toxicity in a subject comprising:

(a1) (i) selecting a male or female subject;
(ii) bringing said subject into contact with a compound suspected to be capable of inducing eye toxicity, or
(a2) selecting a male or female subject brought into contact with a compound capable of inducing eye toxicity;
(b) obtaining a test sample from said selected subject;
(c) pre-treating said sample in preparation for analysis;
(d) determining the amount of at least one biomarker selected from any one of Tables 9a, 9b, 9c or 9d in said test sample, and
(e) comparing the amounts determined in step (d) to a reference; and
(f) based on the comparison of step (e), identifying whether the compound is capable of inducing eye toxicity, or not.

In a preferred embodiment of the aforementioned method said compound is at least one compound selected from the group consisting of: [3-(4,5-dihydro-isoxazol-3-yl)-4-methylsulfonyl-2-chlor phenyl](5-hydroxy-1-methyl-1H-pyrazol-4-yl)methanone and NTBC (HPPD-Inhibitor).

In another preferred embodiment of the methods of the present invention said reference is derived from (i) a subject or group of subjects which suffers from eye toxicity or (ii) a subject or group of subjects which has been brought into contact with at least one compound selected from the group consisting of: [3-(4,5-dihydro-isoxazol-3-yl)-4-methylsulfonyl-2-chlor phenyl](5-hydroxy-1-methyl-1H-pyrazol-4-yl)methanone and NTBC (HPPD-Inhibitor). In a more preferred embodiment of said method essentially identical amounts for the biomarkers in the test sample and the reference are indicative for eye toxicity.

In another preferred embodiment of the methods of the present invention said reference is derived from (i) a subject or group of subjects known to not suffer from eye toxicity or (ii) a subject or group of subjects which has not been brought into contact with at least one compound selected from the group consisting of: [3-(4,5-dihydro-isoxazol-3-yl)-4-methylsulfonyl-2-chlor phenyl](5-hydroxy-1-methyl-1H-pyrazol-4-yl)methanone and NTBC (HPPD-Inhibitor). In a more preferred embodiment of said methods amounts for the biomarkers which differ in the test sample in comparison to the reference are indicative for eye toxicity.

In yet another embodiment of the methods of the present invention said reference is a calculated reference for the biomarkers for a population of subjects. In a more preferred embodiment of said methods amounts for the biomarkers which differ in the test sample in comparison to the reference are indicative for eye toxicity.

The present invention also contemplates a method of identifying a substance for treating eye toxicity comprising the steps of:

(a) determining in a sample of a subject suffering from eye toxicity which has been brought into contact with a candidate substance suspected to be capable of treating eye toxicity the amount of at least one biomarker selected from any one of Tables 9a, 9b, 9c, or 9d; and
(b) comparing the amounts determined in step (a) to a reference, whereby a substance capable of treating eye toxicity is to be identified.

In a particular embodiment of the method of the invention, a method is provided for identifying a substance for treating eye toxicity comprising:

(a1) (i) selecting a male or female subject;
(ii) bringing said subject into contact with a compound suspected to be capable of inducing eye toxicity such that eye toxicity is elicited, or
(a2) selecting a male or female suffering from eye toxicity;
(b) obtaining a test sample from said selected subject;
(c) pre-treating said sample in preparation for analysis;
(d) determining the amount of at least one biomarker selected from any one of Tables 9a, 9b, 9c or 9d in said test sample, and
(e) comparing the amounts determined in step (d) to a reference; and
(f) based on the comparison of step (e), identifying and selecting the substance for treating eye toxicity.

In a preferred embodiment of the aforementioned method said reference is derived from (i) a subject or group of subjects which suffers from eye toxicity or (ii) a subject or group of subjects which has been brought into contact with at least one compound selected from the group consisting of: [3-(4,5-dihydro-isoxazol-3-yl)-4-methylsulfonyl-2-chlor phenyl](5-hydroxy-1-methyl-1H-pyrazol-4-yl)methanone and NTBC (HPPD-Inhibitor). In a more preferred embodiment of said method amounts for the biomarkers which differ in the test sample and the reference are indicative for a substance capable of treating eye toxicity.

In another preferred embodiment of the aforementioned method said reference is derived from (i) a subject or group of subjects known to not suffer from eye toxicity or (ii) a subject or group of subjects which has not been brought into contact with at least one compound selected from the group consisting of [3-(4,5-dihydro-isoxazol-3-yl)-4-methylsulfonyl-2-chlor phenyl](5-hydroxy-1-methyl-1H-pyrazol-4-yl)methanone and NTBC (HPPD-Inhibitor). In a more preferred embodiment of the said methods essentially identical amounts for the biomarkers in the test sample and the reference are indicative for a substance capable of treating eye toxicity.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating eye toxicity.

The present invention also relates to the use of at least one biomarker selected from anyone of Tables 9a, 9b, 9c, or 9d or a detection agent for the said biomarker for diagnosing eye toxicity in a sample of a subject.

Moreover, the present invention relates to a device for diagnosing eye toxicity in a sample of a subject suspected to suffer therefrom comprising:

(a) an analyzing unit comprising a detection agent for at least one biomarker selected from any one of Tables 9a, 9b, 9c, or 9d which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby eye toxicity is diagnosed.

In a preferred embodiment of the device of the invention said stored reference is a reference derived from a subject or a group of subjects known to suffer from eye toxicity or a subject or group of subjects which has been brought into contact with at least one compound selected from the group consisting of [3-(4,5-dihydro-isoxazol-3-yl)-4-methylsulfonyl-2-chlor phenyl](5-hydroxy-1-methyl-1H-pyrazol-4-yl)methanone and NTBC (HPPD-Inhibitor), and said data processor executes instructions for comparing the amount of the at least one biomarker determined by the analyzing unit to the stored reference, wherein an essentially identical amount of the at least one biomarker in the test sample in comparison to the reference is indicative for the presence of eye toxicity or wherein an amount of the at least one biomarker in the test sample which differs in comparison to the reference is indicative for the absence of eye toxicity.

In another preferred embodiment of the device of the invention said stored reference is a reference derived from a subject or a group of subjects known to not suffer from eye toxicity or a subject or group of subjects which has not been brought into contact with at least one compound selected from the group consisting of [3-(4,5-dihydro-isoxazol-3-yl)-4-methylsulfonyl-2-chlor phenyl](5-hydroxy-1-methyl-1H-pyrazol-4-yl)methanone and NTBC (HPPD-Inhibitor), and said data processor executes instructions for comparing the amount of the at least one biomarker determined by the analyzing unit to the stored reference, wherein an amount of the at least one biomarker in the test sample which differs in comparison to the reference is indicative for the presence of eye toxicity or wherein an essential identical amount of the at least one biomarker in the test sample in comparison to the reference is indicative for the absence of eye toxicity.

Further, the present invention relates to a kit for diagnosing eye toxicity comprising a detection agent for the at least one biomarker selected from any one of Tables 9a, 9b, 9c, or 9d and standards for the at least one biomarker the concentration of which is derived from a subject or a group of subjects known to suffer from eye toxicity or derived from a subject or a group of subjects known to not suffer from eye toxicity.

In particular, the present invention relates to a method for diagnosing skeletal muscle innervation stimulation comprising:

(a) determining the amount of at least one biomarker selected from any one of Tables 12a or 12b in a test sample of a subject suspected to suffer from said skeletal innervation stimulation, and
(b) comparing the amounts determined in step (a) to a reference, whereby skeletal innervation stimulation is to be diagnosed.

In a particular embodiment of the method of the invention, a method is provided for diagnosing skeletal muscle innervation stimulation comprising:

(a) selecting a male or female subject suspected to suffer from skeletal muscle innervation stimulation;
(b) obtaining a test sample from said selected subject;
(c) pre-treating said sample in preparation for analysis;
(d) determining the amount of at least one biomarker selected from any one of Tables 12a or 12b in said test sample, and
(e) comparing the amounts determined in step (d) to a reference; and
(f) based on the comparison of step (e), diagnose skeletal muscle innervation stimulation by monitoring, confirmation or classification of the skeletal muscle innervation stimulation or its symptoms.

In a preferred embodiment of the aforementioned method said subject has been brought into contact with a compound suspected to be capable of skeletal muscle innervation stimulation.

The present invention also relates to a method of determining whether a compound is capable of inducing skeletal muscle innervation stimulation in a subject comprising:

(a) determining in a sample of a subject which has been brought into contact with a compound suspected to be capable of inducing skeletal muscle innervation stimulation the amount of at least one biomarker selected from any one of Tables 12a or 12b; and
(b) comparing the amounts determined in step (a) to a reference, whereby the capability of the compound to induce skeletal muscle innervation stimulation is determined.

In a particular embodiment of the method of the invention, a method is provided for determining whether a compound is capable of inducing skeletal muscle innervation stimulation in a subject comprising:

(a1) (i) selecting a male or female subject;
(ii) bringing said subject into contact with a compound suspected to be capable of inducing skeletal muscle innervation stimulation, or
(a2) selecting a male or female subject brought into contact with a compound capable of inducing skeletal muscle innervation stimulation;
(b) obtaining a test sample from said selected subject;
(c) pre-treating said sample in preparation for analysis;
(d) determining the amount of at least one biomarker selected from any one of Tables 12a or 12b in said test sample, and
(e) comparing the amounts determined in step (d) to a reference; and
(f) based on the comparison of step (e), identifying whether the compound is capable of inducing skeletal muscle innervation stimulation, or not.

In a preferred embodiment of the aforementioned method said compound is at least one compound selected from the group consisting of: Toxaphene, Glipizide, Lead acetate trihydrate, and Thallium(I) acetate.

In another preferred embodiment of the methods of the present invention said reference is derived from (i) a subject or group of subjects which suffers from skeletal muscle innervation stimulation or (ii) a subject or group of subjects which has been brought into contact with at least one compound selected from the group consisting of: Toxaphene, Glipizide, Lead acetate trihydrate, and Thallium(I) acetate. In a more preferred embodiment of said method essentially identical amounts for the biomarkers in the test sample and the reference are indicative for skeletal muscle innervation stimulation.

In another preferred embodiment of the methods of the present invention said reference is derived from (i) a subject or group of subjects known to not suffer from skeletal muscle innervation stimulation or (ii) a subject or group of subjects which has not been brought into contact with at least one compound selected from the group consisting of: Toxaphene, Glipizide, Lead acetate trihydrate, and Thallium(I) acetate. In a more preferred embodiment of said methods amounts for the biomarkers which differ in the test sample in comparison to the reference are indicative for skeletal muscle innervation stimulation.

In yet another embodiment of the methods of the present invention said reference is a calculated reference for the biomarkers for a population of subjects. In a more preferred embodiment of said methods amounts for the biomarkers which differ in the test sample in comparison to the reference are indicative for skeletal muscle innervation stimulation.

The present invention also contemplates a method of identifying a substance for treating skeletal muscle innervation stimulation comprising the steps of:

(a) determining in a sample of a subject suffering from skeletal muscle innervation stimulation which has been brought into contact with a candidate substance suspected to be capable of treating skeletal muscle innervation stimulation the amount of at least one biomarker selected from any one of Tables 12a or 12b; and
(b) comparing the amounts determined in step (a) to a reference, whereby a substance capable of treating skeletal muscle innervation stimulation is to be identified.

In a particular embodiment of the method of the invention, a method is provided for identifying a substance for treating skeletal muscle innervation stimulation comprising:

(a1) (i) selecting a male or female subject;
(ii) bringing said subject into contact with a compound suspected to be capable of inducing muscle innervation stimulation such that muscle innervation stimulation is elicited, or
(a2) selecting a male or female suffering from muscle innervation stimulation;
(b) obtaining a test sample from said selected subject;
(c) pre-treating said sample in preparation for analysis;
(d) determining the amount of at least one biomarker selected from any one of Tables 12a or 12b in said test sample, and
(e) comparing the amounts determined in step (d) to a reference; and
(f) based on the comparison of step (e), identifying and selecting the substance for treating muscle innervation stimulation.

In a preferred embodiment of the aforementioned method said reference is derived from (i) a subject or group of subjects which suffers from skeletal muscle innervation stimulation or (ii) a subject or group of subjects which has been brought into contact with at least one compound selected from the group consisting of: Toxaphene, Glipizide, Lead acetate trihydrate, and Thallium(I) acetate. In a more preferred embodiment of said method amounts for the biomarkers which differ in the test sample and the reference are indicative for a substance capable of treating skeletal muscle innervation stimulation.

In another preferred embodiment of the aforementioned method said reference is derived from (i) a subject or group of subjects known to not suffer from skeletal muscle innervation stimulation or (ii) a subject or group of subjects which has not been brought into contact with at least one compound selected from the group consisting of: Toxaphene, Glipizide, Lead acetate trihydrate, and Thallium(I) acetate. In a more preferred embodiment of the said methods essentially identical amounts for the biomarkers in the test sample and the reference are indicative for a substance capable of treating skeletal muscle innervation stimulation.

In yet another preferred embodiment of the aforementioned method said reference is a calculated reference for the biomarkers in a population of subjects. In a more preferred embodiment of the said methods essentially identical amounts for the biomarkers in the test sample and the reference are indicative for a substance capable of treating skeletal muscle innervation stimulation.

The present invention also relates to the use of at least one biomarker selected from any one of Tables 12a or 12b or a detection agent for the said biomarker for diagnosing skeletal muscle innervation stimulation in a sample of a subject.

Moreover, the present invention relates to a device for diagnosing skeletal muscle innervation stimulation in a sample of a subject suspected to suffer therefrom comprising:

(a) an analyzing unit comprising a detection agent for at least one biomarker selected from any one of Tables 12a or 12b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby skeletal muscle innervation stimulation is diagnosed.

In a preferred embodiment of the device of the invention said stored reference is a reference derived from a subject or a group of subjects known to suffer from skeletal muscle innervation stimulation or a subject or group of subjects which has been brought into contact with at least one compound selected from the group consisting Toxaphene, Glipizide, Lead acetate trihydrate, and Thallium(I) acetate, and said data processor executes instructions for comparing the amount of the at least one biomarker determined by the analyzing unit to the stored reference, wherein an essentially identical amount of the at least one biomarker in the test sample in comparison to the reference is indicative for the presence of skeletal muscle innervation stimulation or wherein an amount of the at least one biomarker in the test sample which differs in comparison to the reference is indicative for the absence of skeletal muscle innervation stimulation.

In another preferred embodiment of the device of the invention said stored reference is a reference derived from a subject or a group of subjects known to not suffer from skeletal muscle innervation stimulation or a subject or group of subjects which has not been brought into contact with at least one compound selected from the group consisting of Toxaphene, Glipizide, Lead acetate trihydrate, and Thallium(I) acetate, and said data processor executes instructions for comparing the amount of the at least one biomarker determined by the analyzing unit to the stored reference, wherein an amount of the at least one biomarker in the test sample which differs in comparison to the reference is indicative for the presence of skeletal muscle innervation stimulation or wherein an essential identical amount of the at least one biomarker in the test sample in comparison to the reference is indicative for the absence of skeletal muscle innervation stimulation.

Further, the present invention relates to a kit for diagnosing skeletal muscle innervation stimulation comprising a detection agent for the at least one biomarker selected from any one of Tables 12a or 12b and standards for the at least one biomarker the concentration of which is derived from a subject or a group of subjects known to suffer from skeletal muscle innervation stimulation or derived from a subject or a group of subjects known to not suffer from skeletal muscle innervation stimulation.

In particular the present invention contemplates also the following specific methods, uses, devices and kits.

The following definitions and explanations apply mutatis mutandis to all the previous embodiments of the present invention as well as the embodiments described in the following.

The methods referred to in accordance with the present invention may essentially consist of the aforementioned steps or may include further steps. Further steps may relate to sample pretreatment or evaluation of the diagnostic results obtained by the methods. Preferred further evaluation steps are described elsewhere herein. The methods may partially or entirely be assisted by automation. For example, steps pertaining to the determination of the amount of a biomarker can be automated by robotic and automated reader devices. Likewise, steps pertaining to a comparison of amounts can be automated by suitable data processing devices, such as a computer, comprising a program code which when being executed carries out the comparison automatically. A reference in such a case will be provided from a stored reference, e.g., from a database. It is to be understood that the method is, preferably, a method carried out ex vivo on a sample of a subject, i.e. not practised on the human or animal body.

The term “diagnosing” as used herein refers to assessing the probability according to which a subject is suffering from a condition, such as intoxication, disease or disorder referred to herein, or has a predisposition for such a condition. Diagnosis of a predisposition may sometimes be referred to as prognosis or prediction of the likelihood that a subject will develop the condition within a predefined time window in the future. As will be understood by those skilled in the art, such an assessment, although preferred to be, may usually not be correct for 100% of the subjects to be diagnosed. The term, however, requires that a statistically significant portion of subjects can be identified as suffering from the condition or having a predisposition for the condition. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2, 0.1, 0.05.

Diagnosing according to the present invention also includes monitoring, confirmation, and classification of a condition or its symptoms as well as a predisposition therefor. Monitoring refers to keeping track of an already diagnosed condition or predisposition. Monitoring encompasses, e.g., determining the progression of the condition or predisposition, determining the influence of a particular treatment on the progression of the condition or the influence of prophylactic measures such as a prophylactic treatment or diet on the development of the condition in a subject having a predisposition. Said treatment, prophylactic measure or diet may be adjusted and the influence of the adjustment may be investigated as an aspect of the monitoring. Moreover, if progression of the condition or a predisposition therefor is monitored, said monitoring may also include determining a monitoring frequency and to recommend and/or carry out additional monitoring measures such as measurement of additional biochemical or other health parameters. Confirmation relates to the strengthening or substantiating a diagnosis of the condition or a predisposition for the condition already determined using other indicators or markers. Confirmation may also include in an aspect the administration or adaptation of therapeutic measures based on the confirmed condition or predisposition therefor. Classification relates to (i) allocating the condition into different classes, e.g., corresponding to the strength of the symptoms accompanying the condition, or (ii) differentiating between different stages, disease or disorders accompanying the condition. Classification may also include in an aspect the administration or adaptation of therapeutic measures based on the classified condition, symptoms or predisposition therefor. A predisposition for the condition can be classified based on the degree of the risk, i.e. the probability according to which a subject will develop the condition later. Moreover, classification also, preferably, includes allocating a mode of action to a compound to be tested by the methods of the present invention. Specifically, the methods of the present invention allow for determination of a specific mode of action of a compound for which such mode of action is not yet known. This is, preferably, achieved by comparing the amount determined for the at least one biomarker or a biomarker profile representative for said compound to the amount of the biomarker or biomarker profile determined for a compound for which the mode of action is known as a reference. The classification of the mode of action allows an even more reliable assessment of toxicity of a compound because the molecular targets of the compound are identified. The methods of the present invention aiming at diagnosing a disease or condition may be used for screening compounds for toxicological effects and reporting thereon as well as in compound development, e.g., in increasing safety or in developing drugs or identifying effective concentrations.

In accordance with the present invention, a compound can also be identified as being capable of inducing neuronal toxicity. Such identification, preferably, also includes making suggestions for the manufacture, handling, storage and/or transport of the compound and its applications. Such suggestions include establishing safety protocols for manufacture, handling, storage, transport and/or application, labelling the compound according to its toxicity potential, limiting exposure to humans, animals and/or to the environment. Moreover, if a compound is identified as eliciting neuronal toxicity, safety levels such as LD50/LC50 and/or ED50/EC50 values and derived thresholds are, preferably, determined.

The term “neuronal toxicity” as used herein relates to any damage or impairment of neuronal tissue or neuronal derived tissues. In particular, envisaged according to the present invention is toxicity of the central nervous system (“CNS toxicity”) or toxicity of the eye (“eye toxicity”). Moreover, neuronal toxicity also includes aspects of peripheral neuronal toxicity and, in particular, impaired neuromuscular transmission associated with skeletal muscle innervation stimulation. Accordingly, the term neuronal toxicity as used herein encompasses CNS toxicity, eye toxicity and peripheral neuronal toxicity, preferably, associated with skeletal muscle innervation stimulation, in general. Preferably, neuronal toxicity as used herein is induced by or is the result of the administration of a chemical compound or drug, i.e. so-called toxin-induced neuronal toxicity.

The symptoms and clinical signs of the aforementioned manifestations of neuronal toxicity are well known to the person skilled in the art and are described in detail in standard books of toxicology, e.g., H. Marquardt, S. G. Schafer, R. O. McClellan, F. Welsch (eds.), “Toxicology”, Chapter 13: The Liver, 1999, Academic Press, London.

CNS toxicity as used herein, preferably, refers to impaired neuronal function in the central nervous system. Normal functioning of the nervous system is dependent on the innate ability of neurons to express excitability. Transmission of information is reliant on changes in the frequency and regularity of the generation of action potentials. Between neurons, and between neurons and receptor cells, there are synaptic junctions in which transmission occurs by the release of neurotransmitter. Some transmitters are excitatory others are inhibitory. This allows fine control over neuronal activity. The brain may be divided also on a functional basis into various pathways containing cell groups and their axonal connections which utilize the same neurotransmitter. Five major pathways are recognized: (1) Cholinergic pathways of the basal part of the forebrain are concerned with memory, behaviour and mood, and those of the reticular formation of the brainstem influence the state of arousal and control sleep processes. (2) Noradrenergic pathways are restricted to the pontine and medullary regions but have connections with many regions of the central nervous system, including the limbic system. (3) Adrenergic neurons are confined to the caudal part of the hindbrain and the pathways are poorly understood. (4) Serotonergic pathways are found throughout the brainstem and regulate cardiovascular function, particularly cerebral blood flow, and control sleep processes. (5) Dopaminergic pathways are located in the mesencephalon and the hypothalamus. Neurotransmitters which are involved in the transmission of in the CNS include These include the substances, norepinephrine (NE), dopamine (DA), 5-hydroxytryptamine (serotonin, 5HT), acetylcholine (ACh), y-aminobutyric acid (GABA), phenylethylamine, histamine, glycine, glutamic acid, aspartic acid, taurine, gamma amino butyric acid (GABA), and a number of peptides such as substance P, and the enkephalins. Dopamine is a catecholamine neurotransmitter present in a wide variety of animals. In the brain, this substituted phenethylamine activates the five known types of dopamine receptors D1, D2, D3, D4, and D5 and their variants. Dopamine is produced in several areas of the brain and is also a neurohormone released by the hypothalamus. Dopamine has many functions in the brain, including important roles in behavior and cognition, voluntary movement, motivation, punishment and reward, inhibition of prolactin production, sleep, mood, attention, working memory, and learning. These functions may become impaired if the dopamine dependent transmission becomes impaired by intoxication. Glutamate is the main excitatory neurotransmitter of the brain and its effects are mediated by several subtypes of receptors called excitatory amino acid receptors (EAARs) those that are ligand-gated directly to ion channels (ionotropic) and those that are coupled with G proteins. The entry of glutamate into the CNS is regulated at the blood-brain barrier. Glutamate receptors mediate most of the excitatory neurotransmission in the mammalian central nervous system (CNS). They also participate in plastic changes of synaptic transmission underlying long-term potentiation in memory and learning, and the formation of neural networks during development. These functions may become impaired if the glutamate dependent transmission becomes impaired by intoxication. Serotonin (5-hydroxytryptamine, 5-HT) modulates neural activity and a wide range of neuropsychological processes. However, most serotonin is found outside the central nervous system (gastrointestinal tract, platelets), and all serotonin receptors are expressed outside as well as within the brain. Serotonin regulates numerous biological processes including cardiovascular, pulmonary, gastrointestinal (GI), and genitourinary systems as well as the central nervous system (CNS) and dysregulation has been implicated in the pathogenesis of many psychiatric and neurological disorders. Serotonin modulates virtually all human behavioral processes. These functions may become impaired if the 5-HT dependent transmission becomes impaired by intoxication. Norepinephrine or noradrenaline is synthesized from dopamine by dopamine β-hydroxylase. It is a catecholamine with multiple functions including hormone and a neurotransmitter in the central nervous system and sympathetic nervous system released from noradrenergic neurons. Norepinephrine affects parts of the brain, where attention and responses are controlled and underlies the fight-or-flight response, directly increasing heart rate/blood pressure, triggering the release of glucose from energy stores, and increasing blood flow to skeletal muscle and increases the brain's oxygen supply. These functions may become impaired if the norepinehrine dependent transmission becomes impaired by intoxication. Nicotine (methylpyridylpyrrolidine) exerts its effect by binding to certain types of cholinergic receptors, the nicotinic receptors, at synapses in the central nervous system, the peripheral ganglia and the neuromuscular junctions, where it acts as an acetylcholine (AC) agonist. In small concentrations, nicotine increases the activity of these receptors. Nicotine also has effects on a variety of other neurotransmitters. Nicotine has a higher affinity for acetylcholine receptors in the brain than those in skeletal muscle, though at toxic doses it can induce contractions and respiratory paralysis. Clinical effects include sweating, tachycardia, elevated blood pressure and constriction of cutaneous blood vessels. Nicotine in the appropriate area of the CNS has psychoactive actions. It directly stimulates the nicotine subset of CNS and peripheral AC receptors. Moderate poisoning produce symptoms of cholinergic excess including miosis, salivation, urination, defecation, emesis, and increased pulmonary secretions. In large-dose exposure a short live stimulation is followed rapidly by neuromuscular blockade related to persistent membrane depolarization. If death occurs, the most common mechanism is respiratory arrest due to peripheral neuromuscular blockade and cardiovascular collapse. These functions may become impaired if the nicotine dependent transmission becomes impaired by intoxication. As discussed before, an exposure to a variety of drugs and toxins can induce an adverse change in the structure or function of the central nervous system and interpretation of morphological and functional CNS disturbances due to impaired neurotransmitter action in a toxicological setting may be quite complex and may involve both local as well as systemic manifestations of toxicity and/or pharmacologic response. In a general way, these changes can be classified as either quantitative or qualitative. A large number of naturally occurring toxins, as well as many synthetic molecules, cause neurotoxicity by interfering with the processes of chemical transmission between neurons, or between neurons and other cell types, notably skeletal muscle cells. This interference may block either the transmission of impulses or result in accentuation of the neurotransmission, depending on the mechanism of action and on the type of synapse affected. The effect may be transient or permanent. Compounds with similar actions usually have similar chemical and structural properties, and act only on one particular type of synapse. Mechanisms of action differ: some compounds act as agonists or antagonists to the chemical transmitter, while others act by preventing synthesis or release of the neurotransmitter, or by preventing its inactivation or resorption after release.

Preferably, the at least one biomarker to be determined by the methods of the present invention is selected from any one of Tables 1a, 1b, 1c, 1d, 2a, 2b, 3a, 3b, 4a, 4b, 5a, 5b, 6a, 6b, 6c, 6d, 7a, 7b, 8a or 8b if the neuronal toxicity is CNS toxicity. More preferably, said CNS toxicity is associated with impaired GABA dependent transmission, dopamine dependent transmission, and/or serotonine dependent transmission.

More preferably, said CNS toxicity is characterized by impaired GABA dependent transmission if the at least one biomarker is selected from the biomarkers shown in Table 1a, 1b, 1c or 1d.

More preferably, said CNS toxicity is characterized by impaired dopamine dependent transmission if the at least one biomarker is selected from the biomarkers shown in Table 3a, 3b, 4a, 4b, 6a, 6b, 6c, or 6d.

Also more preferably, said CNS toxicity is characterized by an impaired serotonie dependent transmission level if the at least one biomarker is selected from the biomarkers shown in Table 5a, 5b, 7a, 7b, 8a, 8b, 8c or 8d.

More preferably, said CNS toxicity is associated with a pschyoanaleptic effect if the at least one biomarker is selected from the biomarkers shown in Table 2a or 2b.

Eye toxicity as used herein, preferably, refers to an impairment of the function of the eye. The eye is also a structure of the CNS and can be effected by CNS toxicity as well. It is a complex organ but it has one function only, namely photosensory reception. Preferred consequences of eye toxicity include corneal injury from systemic administration may take the form of keratitis, edema or opacification from the deposition of endogenous or exogenous substances. Reported examples of keratitis in this type of injury are few, although the administration of tyrosine in a low protein diet is reported to induce keratitis in rats. Another consequences arises from impaired tyrosine metabolism. Tyrosine has a special role by virtue of the phenol functionality aside from being a proteogenic amino acid. It occurs in proteins that are part of signal transduction processes. It functions as a receiver of pho groups that are transferred by way of protein kinases (so-called receptor tyrosine kinases). Phosphorylation of the hydroxyl group changes the activity of the target protein. Interest in the effects of excessive intakes of tyrosine on experimental animals has as its basis the need to understand the changes in tissue and in metabolism of tyrosine in individuals with the inherited disorder of tyrosine metabolism, tyrosinemia II. Tyrosinemia II is inherited as an autosomal recessive trait and involves a complete lack of the liver enzyme tyrosine aminotransferase associated with tyrosinemia, tyrosinuria, and increased urinary excretion of tyrosine metabolites. Individuals with this disease have corneal lesions and palm and sole erosions in the early months of life. Major progress in understanding the disease has come from observation of laboratory rats fed low-protein diets containing high levels of tyrosine and from studies of mink that have an inherited disorder of tyrosine metabolism. High concentration of plasma and tissue tyrosine causes eye and paw lesions. Within 24 hours of the initiation of feeding diets containing 5% of tyrosine to laboratory rats, alterations appear in the cornea, which are described as dots that progress into “snow flake” opacity. The cellular architecture becomes distorted, the cornea thickens and becomes opaque. The positive identification of crystals within the cells of the cornea of rats fed low-protein diets containing tyrosine has been made. Factors that prevent tyrosine toxicity in rats also prevent crystal formation and corneal lesions. In addition, rats exposed to sulcotrione revealed corneal lesions that varied in severity from partial or hazy opacity to complete opacity, with edema and neovascularization evident in the more extensive lesions. Tyrosine concentration could be as much as 10 times the normal value after 24 hours when rats were given the sulcotrione analogue in a single dose. It also caused cornea damage. Corneal opacity in individual rats was also found, which might be related to the accumulation of excessive tyrosine in the anterior chamber of eye. Interpretation of eye changes with regards to tyrosine in a toxicological setting may be quite complex as the heterogeneous nature of the eye and its composition of different tissue types renders it particularly susceptible to toxicity. This spectrum of anatomical and functional features provides a wide variety of pathological responses which are important in toxicology. The eye is notorious for demonstrating marked species variation in its response to toxic chemicals. This, together with the generally poor understanding of mechanisms with respect to ocular toxicity, renders the extrapolation of the effects of toxic chemicals in laboratory animals to man extremely difficult. To make broad generalizations on this subject is fraught with hazard, except to regard experimental oculotoxicity testing as being of value only to identify the potential of a chemical to cause ocular damage in man.

Preferably, the at least one biomarker to be determined by the methods of the present invention is selected from any one of Tables 9a, 9b, 9c, or 9d if the neuronal toxicity is eye toxicity.

More preferably, said eye toxicity is associated with inhibition of hallucinogen persisting perception disorder (HPPD) if the at least one biomarker is selected from the biomarkers shown in Table 9. Preferably, the at least one biomarker to be determined by the methods of the present invention is selected from any one of Tables 12a or 12b if the neuronal toxicity is skeletal muscle innervation stimulation.

The term “skeletal muscle innervation stimulation” as referred to in accordance with the present invention encompasses non-physiological and improper stimulation of skeletal muscle activity caused by impaired neuromuscular transmission. Said non-physiological and improper stimulation of skeletal muscle activity, preferably, results in an impaired metabolism of the muscle cells and, in particular, their energy deposits.

It was found in accordance with the present invention that a combination of more than one of the biomarkers listed in the Tables further strengthen the diagnosis since each of the biomarkers is an apparently statistically independent predictor for the diagnosis. Moreover, the specificity for neuronal toxicity is also significantly increased since influences from other tissues on the marker abundance are counterbalanced. Thus, the term “at least one” as used herein, preferably, refers to a combination of at least 2, at least 3, at least 4, at least 5, at least 6, at least 7, at least 8, at least 9 or at least 10 of the biomarkers referred to in any one of the accompanying Tables. Preferably, all biomarkers recited in any one of the Tables are to be determined in combination in accordance with the methods of the present invention.

Preferred groups or combinations of biomarkers for neuronal toxicity from the individual tables and for the indications referred to in the tables are as follows:

### Tables 1a and 1b (CNS GABA Receptor Agonist):

Phosphate (inorganic and organic phosphates), Phosphatidylcholine (C16:0,C22:6), Phosphatidylcholine (C16:0,C20:4), Cysteine or 3-Indoxylsulfate.

### Tables 1c and 1d (CNS GABA Receptor Agonist):

Glutamate, Cysteine, Coenzyme Q9, myo-Inositol or Isoleucine.

### Tables 2a and 2b (CNS Psycoanaleptics):

Choline plasmalogen No 03, DAG (C18:1,C18:2), Glutamate, Isoleucine or TAG No 07.

### Tables 3a and 3b (CNS Antipsychotic Drugs (Dopamine Receptor Block):

Threonic acid, myo-Inositol-2-phosphate, lipid fraction, Tryptophan, Glycine or 3-Methoxytyrosine.

### Tables 4a and 4b (CNS Dopamine Antagonists):

Phosphatidylcholine (C16:0,C16:0), Docosapentaenoic acid (C22:cis[7,10,13,16,19]5), Phosphatidylcholine (C16:1,C18:2), Ceramide (d18:1,C24:1) or myo-Inositol, lipid fraction.

### Tables 5a and 5b (CNS Receptor Block (Serotonine-Dopamine):

Glutamine, Galactose, lipid fraction, Valine, Leucine or Isoleucine.

### Tables 6a and 6b (Nerve System Dopamin Agonist):

Ceramide (d18:1,C24:1), 16-Methylheptadecanoic acid, Homovanillic acid (HVA), Choline plasmalogen No 03 or 17-Methyloctadecanoic acid.

### Tables 6c and 6d (Nerve System Serotonin Receptor Antagonist):

Docosapentaenoic acid (C22:cis[7,10,13,16,19]5), Phosphatidylcholine (C16:0,C16:0), Phosphatidylcholine (C16:0,C22:6), Docosahexaenoic acid (C22:cis[4,7,10,13,16,19]6) or Phosphatidylcholine No 02.

### Tables 7a and 7b (Nerve System Serotonin Reuptake Inhibition):

Serotonin (5-HT), 4-Hydroxysphinganine (t18:0, Phytosphingosine), total, Adrenaline (Epinephrine), Threonine or Glutamate.

### Tables 8a and 8b (Nerve System Serotonine Reuptake Inhibition):

4-Hydroxysphinganine (t18:0, Phytosphingosine), total, DAG (C18:1,C18:2), Serotonin (5-HT), Ceramide (d18:1,C24:1) or threo-Sphingosine.

### Tables 8c and 8d (Nervous System Dopamin Agonist):

16-Methylheptadecanoic acid, Ceramide (d18:1,C24:1), Phosphatidylcholine (C16:0,C20:4), Pyruvate or 17-Methyloctadecanoic acid.

### Tables 9a and 9b (Eye HPPD Inhibition):

4-Hydroxyphenylpyruvate, Glutamine, 5-Oxoproline, Tyrosine or Glycine.

### Tables 9c and 9d (Eye HPPD Inhibition):

Tyrosine, Threonine, Glutamine, Lysine or Citrulline.

Thus, preferably, the at least one biomarker is at least one biomarker selected from the aforementioned group or the at least one biomarker is a combination of biomarkers consisting or comprising the aforementioned group of biomarkers. The aforementioned biomarkers and combinations of biomarkers have been identified as key biomarkers having a particular high diagnostic value as described in more detail in the accompanying Examples.

Furthermore, other biomarkers or clinical parameters including known metabolites, genetic mutations, transcript and/or protein amounts or enzyme activities may still be determined in addition. Such, additional clinical or biochemical parameters which may be determined in accordance with the method of the present invention are well known in the art.

The term “biomarker” as used herein refers to a chemical compound whose presence or concentration in a sample is indicative for the presence or absence or strength of a condition, preferably, neuronal toxicity as referred to herein. The chemical compound is, preferably, a metabolite or an analyte derived therefrom. An analyte is a chemical compound which can be identical to the actual metabolite found in an organism. However, the term also includes derivatives of such metabolites which are either endogenously generated or which are generated during the isolation or sample pre-treatment or as a result of carrying out the methods of the invention, e.g., during the purification and/or determination steps. In specific cases the analyte is further characterized by chemical properties such as solubility. Due to the said properties, the analyte may occur in polar or lipid fractions obtained during the purification and/or determination process. Thus, chemical properties and, preferably, the solubility shall result in the occurrence of an analyte in either polar or lipid fractions obtained during the purification and/or determination process. Accordingly, the said chemical properties and, in particular the solubility taken into account as the occurrence of an analyte in either polar or lipid fractions obtained during the purification and/or determination process shall further characterize the analyte and assist in its identification. Details on how these chemical properties can be determined and taken into account are found in the accompanying Examples described below. Preferably, the analyte represents the metabolite in a qualitative and quantitative manner and, thus, allows inevitably concluding on the presence or absence or the amount of the metabolite in a subject or at least in the test sample of said subject. Biomarker, analyte and metabolite are referred to herein in the singular but also include the plurals of the terms, i.e. refer to a plurality of biomarker, analyte or metabolite molecules of the same molecular species. Moreover, a biomarker according to the present invention is not necessarily corresponding to one molecular species. Rather, the biomarker may comprise stereoisomers or enantiomers of a compound. Further, a biomarker can also represent the sum of isomers of a biological class of isomeric molecules. Said isomers shall exhibit identical analytical characteristics in some cases and are, therefore, not distinguishable by various analytical methods including those applied in the accompanying Examples described below. However, the isomers will share at least identical sum formula parameters and, thus, in the case of, e.g., lipids an identical chain length and identical numbers of double bonds in the fatty acid and/or sphingo base moieties

The term “test sample” as used herein refers to samples to be used for the diagnosis of neuronal toxicity by the methods of the present invention. Preferably, said test sample is a biological sample. Samples from biological sources (i.e. biological samples) usually comprise a plurality of metabolites. Preferred biological samples to be used in the method of the present invention are samples from body fluids, preferably, blood, plasma, serum, saliva, bile, urine or cerebrospinal fluid, or samples derived, e.g. by biopsy, from cells, tissues or organs, preferably from the liver. More preferably, the sample is a blood, plasma or serum sample, most preferably, a plasma sample. Biological samples are derived from a subject as specified elsewhere herein. Techniques for obtaining the aforementioned different types of biological samples are well known in the art. For example, blood samples may be obtained by blood taking while tissue or organ samples are to be obtained, e.g. by biopsy.

The aforementioned samples are, preferably, pre-treated before they are used for the methods of the present invention. As described in more detail below, said pre-treatment may include treatments required to release or separate the compounds or to remove excessive material or waste. Suitable techniques comprise centrifugation, extraction, fractioning, ultra-filtration, protein precipitation followed by filtration and purification and/or enrichment of compounds. Moreover, other pretreatments are carried out in order to provide the compounds in a form or concentration suitable for compound analysis. For example, if gas-chromatography coupled mass spectrometry is used in the method of the present invention, it will be required to derivatize the compounds prior to the said gas chromatography. Suitable and necessary pre-treatments depend on the means used for carrying out the method of the invention and are well known to the person skilled in the art. Pre-treated samples as described before are also comprised by the term “sample” as used in accordance with the present invention.

The term “subject” as used herein relates to animals, preferably to mammals such as mice, rats, guinea pigs, rabbits, hamsters, pigs, sheep, dogs, cats, horses, monkeys, or cows and, also preferably, to humans. More preferably, the subject is a rodent and, most preferably, a rat. Other animals which may be diagnosed applying the methods of the present invention are fishes, birds or reptiles. Preferably, said subject was in or has been brought into contact with a compound suspected to be capable of inducing neuronal toxicity. A subject which has been brought into contact with a compound suspected to induce neuronal toxicity may, e.g., be a laboratory animal such as a rat which is used in a screening assay for, e.g., toxicity of compounds. A subject suspected to have been in contact with a compound capable of inducing neuronal toxicity may be also a subject to be diagnosed for selecting a suitable therapy. Preferably, a compound capable of inducing neuronal toxicity as used herein is 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, and Thallium(I) acetate.

Preferably, the at least one biomarker to be determined by the methods of the present invention is selected from any one of Tables 1c, 1d, 4a, 4b, 5a, 5b, 6a, 6b, 6c, 6d, 7a, 7b, 8c, 8d, 9c or 9d if the subject is a female.

Preferably, the at least one biomarker to be determined by the methods of the present invention is selected from any one of Tables 1a, 1b, 2a, 2b, 3a, 3b, 8a, 8b, 9a, 9b, 12a or 12b if the subject is a male.

The term “determining the amount” as used herein refers to determining at least one characteristic feature of the biomarker, i.e. the metabolite or analyte. Characteristic features in accordance with the present invention are features which characterize the physical and/or chemical properties including biochemical properties of a biomarker. Such properties include, e.g., molecular weight, viscosity, density, electrical charge, spin, optical activity, colour, fluorescence, chemoluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of a biomarker by standard operations, e.g., mathematical calculations such as multiplication, division or logarithmic calculus. Most preferably, the at least one characteristic feature allows the determination and/or chemical identification of the biomarker and its amount. Accordingly, the characteristic value, preferably, also comprises information relating to the abundance of the biomarker from which the characteristic value is derived. For example, a characteristic value of a biomarker may be a peak in a mass spectrum. Such a peak contains characteristic information of the biomarker, i.e. the m/z (mass to charge ratio) information, as well as an intensity value being related to the abundance of the said biomarker (i.e. its amount) in the sample.

As discussed before, the at least one biomarker to be determined in accordance with the methods of the present invention may be, preferably, determined quantitatively or semi-quantitatively. For quantitative determination, either the absolute or precise amount of the biomarker will be determined or the relative amount of the biomarker will be determined based on the value determined for the characteristic feature(s) referred to herein above. The relative amount may be determined in a case were the precise amount of a biomarker can or shall not be determined. In said case, it can be determined whether the amount in which the biomarker is present is enlarged or diminished with respect to a second sample comprising said biomarker in a second amount. Quantitatively analysing a biomarker, thus, also includes what is sometimes referred to as semi-quantitative analysis of a biomarker.

Moreover, determining as used in the methods of the present invention, preferably, includes using a compound separation step prior to the analysis step referred to before. Preferably, said compound separation step yields a time resolved separation of the at least one biomarker comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or GC are chromatographic techniques to be envisaged by the methods of the present invention. Suitable devices for such determination of biomarkers are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Most preferably, LC-MS and/or GC-MS are used as described in detail below. Said techniques are disclosed in, e.g., Nissen 1995, Journal of Chromatography A, 703: 37-57, U.S. Pat. No. 4,540,884 or U.S. Pat. No. 5,397,894, the disclosure content of which is hereby incorporated by reference. As an alternative or in addition to mass spectrometry techniques, the following techniques may be used for compound determination: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado. The method of the present invention shall be, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches.

Moreover, the biomarker can also be determined by a specific chemical or biological assay. Said assay shall comprise means which allow for specifically detecting the biomarker in the sample. Preferably, said means are capable of specifically recognizing the chemical structure of the biomarker or are capable of specifically identifying the biomarker based on its capability to react with other compounds or its capability to elicit a response in a biological read out system (e.g., induction of a reporter gene). Means which are capable of specifically recognizing the chemical structure of a biomarker are, preferably, detection agents which specifically bind to the biomarker, more preferably, antibodies or other proteins which specifically interact with chemical structures, such as receptors or enzymes, or aptameres. Specific antibodies, for instance, may be obtained using the biomarker as antigen by methods well known in the art. Antibodies as referred to herein include both polyclonal and monoclonal antibodies, as well as fragments thereof, such as Fv, Fab and F(ab)₂ fragments that are capable of binding the antigen or hapten. The present invention also includes humanized hybrid antibodies wherein amino acid sequences of a non-human donor antibody exhibiting a desired antigen-specificity are combined with sequences of a human acceptor antibody. Moreover, encompassed are single chain antibodies. The donor sequences will usually include at least the antigen-binding amino acid residues of the donor but may comprise other structurally and/or functionally relevant amino acid residues of the donor antibody as well. Such hybrids can be prepared by several methods well known in the art. Suitable proteins which are capable of specifically recognizing the metabolite are, preferably, enzymes which are involved in the metabolic conversion of the said biomarker. Said enzymes may either use the biomarker, e.g., a metabolite, as a substrate or may convert a substrate into the biomarker, e.g., metabolite. Moreover, said antibodies may be used as a basis to generate oligopeptides which specifically recognize the biomarker. These oligopeptides shall, for example, comprise the enzyme's binding domains or pockets for the said biomarker. Suitable antibody and/or enzyme based assays may be RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA) or solid phase immune tests. Aptameres which specifically bind to the biomarker can be generated by methods well known in the art (Ellington 1990, Nature 346:818-822; Vater 2003, Curr Opin Drug Discov Devel 6(2): 253-261). Moreover, the biomarker may also be identified based on its capability to react with other compounds, i.e. by a specific chemical reaction. Further, the biomarker may be determined in a sample due to its capability to elicit a response in a biological read out system. The biological response shall be detected as read out indicating the presence and/or the amount of the metabolite comprised by the sample. The biological response may be, e.g., the induction of gene expression or a phenotypic response of a cell or an organism.

The term “reference” refers to values of characteristic features of the at least one biomarker and, preferably, values indicative for an amount of the said biomarker which can be correlated to neuronal toxicity.

Such references are, preferably, obtained from a sample derived from a subject or group of subjects which suffer from neuronal toxicity or from a sample derived from a subject or group of subjects which have/has been brought into contact with 17-alpha-Ethynylestradiol, Apomorphine, 35 [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride Toxaphene, Glipizide, Lead acetate trihydrate or Thallium(I) acetate. A subject or group of subjects may be brought into contact with the said compounds by each topic or systemic administration mode as long as the compounds become bioavailable.

Preferably, the aforementioned compounds can be administered to the subject or the individuals of the group of subjects from which the reference is derived as described in the accompanying Examples and Tables below.

In particular, 17-alpha-Ethynylestradiol, Apomorphine, Bromocriptine Mesylate, Cabergoline, Chlorpromazine, Citalopram hydrobromide, Dextroamphetamine sulfate, Escitalopram oxalate, Fluoxetine hydrochloride, Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, or Ziprasidone hydrochloride as referred to herein are compounds capable of inducing CNS toxicity while [3-(4,5-dihydro-isoxazol-3-yl)-4-methylsulfonyl-2-chlor phenyl](5-hydroxy-1-methyl-1H-pyrazol-4-yl)methanone or NTBC (HPPD-Inhibitor) shall be capable of inducing eye toxicity. Toxaphene, Glipizide, Lead acetate trihydrate, and Thallium(I) acetate are compounds capable of eliciting skeletal muscle innervation stimulation.

Alternatively, but nevertheless also preferred, the reference may be obtained from sample derived from a subject or group of subjects which has not been brought into contact with 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate or Thallium(I) acetate or a healthy subject or group of such subjects with respect to neuronal toxicity and, more preferably, other diseases as well.

The reference may be determined as described hereinabove for the amounts of the biomarkers. In particular, a reference is, preferably, obtained from a sample of a group of subjects as referred to herein by determining the relative or absolute amounts of each of the at least one biomarker(s) in samples from each of the individuals of the group separately and subsequently determining a median or average value for said relative or absolute amounts or any parameter derived therefrom by using statistical techniques referred to elsewhere herein. Alternatively, the reference may be, preferably, obtained by determining the relative or absolute amount for each of the at least one biomarker in a sample from a mixture of samples of the group of subjects as referred to herein. Such a mixture, preferably, consists of portions of equal volume from samples obtained from each of the individuals of the said group.

Moreover, the reference, also preferably, could be a calculated reference, most preferably the average or median value, for the relative or absolute amount for each of the at least one biomarker derived from a population of individuals. Said population of individuals is the population from which the subject to be investigated by the method of the present invention originates. However, it is to be understood that the population of subjects to be investigated for determining a calculated reference, preferably, either consist of apparently healthy subjects (e.g. untreated) or comprise a number of apparently healthy subjects which is large enough to be statistically resistant against significant average or median changes due to the presence of the test subject(s) in the said population. The absolute or relative amounts of the at least one biomarker of said individuals of the population can be determined as specified elsewhere herein. How to calculate a suitable reference value, preferably, the average or median, is well known in the art. Other techniques for calculating a suitable reference include optimization using receiver operating characteristics (ROC) curve calculations which are also well known in the art and which can be performed for an assay system having a given specificity and sensitivity based on a given cohort of subjects without further ado. The population or group of subjects referred to before shall comprise a plurality of subjects, preferably, at least 5, 10, 50, 100, 1,000 or 10,000 subjects up to the entire population. More preferably, the group of subjects referred to in this context is a group of subjects having a size being statistically representative for a given population, i.e. a statistically representative sample. It is to be understood that the subject to be diagnosed by the methods of the present invention and the subjects of the said plurality of subjects are of the same species and, preferably, of the same gender.

More preferably, the reference will be stored in a suitable data storage medium such as a database and are, thus, also available for future diagnoses. This also allows efficiently diagnosing predisposition for neuronal toxicity because suitable reference results can be identified in the database once it has been confirmed (in the future) that the subject from which the corresponding reference sample was obtained (indeed) developed neuronal toxicity.

The term “comparing” refers to assessing whether the amount of the qualitative or quantitative determination of the at least one biomarker is identical to a reference or differs therefrom.

In case the reference results are obtained from a sample derived from a subject or group of subjects suffering from neuronal toxicity or a subject or group of subjects which has been brought into contact with 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate or Thallium(I) acetate, neuronal toxicity can be diagnosed based on the degree of identity or similarity between the amounts obtained from the test sample and the aforementioned reference, i.e. based on an identical qualitative or quantitative composition with respect to the at least one biomarker. Identical amounts include those amounts which do not differ in a statistically significant manner and are, preferably, within at least the interval between 1st and 99th percentile, 5th and 95th percentile, 10th and 90th percentile, 20th and 80th percentile, 30th and 70th percentile, 40th and 60th percentile of the reference, more preferably, the 50th; 60th, 70th, 80th, 90th or 95th percentile of the reference. A reference obtained from a sample derived from a subject or group of subjects suffering from neuronal toxicity or a subject or group of subjects which has been brought into contact with 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, or Thallium(I) acetate, can be applied in the methods of the present invention in order to diagnose neuronal toxicity or for determining whether a compound is capable of inducing neuronal toxicity in a subject. In such a case, preferably, an amount of the at least one biomarker which is essentially identical to the reference will be indicative for the presence of neuronal toxicity or a compound which is capable of inducing neuronal toxicity, while an amount of the at least one biomarker which differs from the reference will be indicative for the absence of neuronal toxicity or a compound which is not capable of inducing neuronal toxicity.

Moreover, a reference obtained from a sample derived from a subject or group of subjects suffering from neuronal toxicity or a subject or group of subjects which has been brought into contact with 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate or Thallium(I) acetate, can be applied for identifying a substance for treating neuronal toxicity. In such a case, preferably, an amount of the at least one biomarker which differs from the reference will be indicative for a substance suitable for treating neuronal toxicity, while an amount of the at least one biomarker which is essentially identical to the reference will be indicative for a substance which is not capable of treating neuronal toxicity.

In case the reference results are obtained from a sample of a subject or group of subjects which has not been brought into contact with 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride; Risperidone, Selegiline hydrochloride, Sertraline hydrochloride, Ziprasidone hydrochloride, Toxaphene, Glipizide, Lead acetate trihydrate or Thallium(I) acetate or which does not suffer from neuronal toxicity, said neuronal toxicity can be diagnosed based on the differences between the test amounts obtained from the test sample and the aforementioned reference, i.e. differences in the qualitative or quantitative composition with respect to the at least one biomarker.

The same applies if a calculated reference as specified above is used.

The difference may be an increase in the absolute or relative amount of the at least one biomarker (sometimes referred to as up-regulation of the biomarker; see also Examples) or a decrease in either of said amounts or the absence of a detectable amount of the biomarker (sometimes referred to as down-regulation of the biomarker; see also Examples). Preferably, the difference in the relative or absolute amount is significant, i.e. outside of the interval between 45^{th} and 55^{th} percentile, 40^{th} and 60^{th} percentile, 30^{th} and 70^{th} percentile, 20^{th} and 80^{th} percentile, 10^{th} and 90^{th} percentile, 5^{th} and 95^{th} percentile, 1^{st} and 99^{th} percentile of the reference.

A reference obtained from a sample derived from a subject or group of subjects which has not been brought into contact with 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate or Thallium(I) acetate or which does not suffer from neuronal toxicity can be applied in the methods of the present invention in order to diagnose the neuronal toxicity or for determining whether a compound is capable of inducing neuronal toxicity in a subject. In such a case, preferably, an amount of the at least one biomarker which differs from the reference will be indicative for the presence of neuronal toxicity or a compound which is capable of inducing neuronal toxicity, while an amount of the at least one biomarker which is essentially identical to the reference will be indicative for the absence of neuronal toxicity or a compound which is not capable of inducing neuronal toxicity. Moreover, a reference obtained from a sample derived from a subject or group of subjects which has not been brought into contact with 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate or Thallium(I) acetate, or which-does not suffer from neuronal toxicity can be applied for identifying a substance for treating neuronal toxicity. In such a case, preferably, an amount of the at least one biomarker which is essentially identical to the reference will be indicative for a substance suitable for treating neuronal toxicity, while an amount of the at least one biomarker which differs from the reference will be indicative for a substance which is not suitable for treating neuronal toxicity.

Preferred references are those referred to in the accompanying Tables or those which can be generated following the accompanying Examples. Moreover, relative differences, i.e. increases or decreases in the amounts for individual biomarkers, are preferably, those recited in the Tables below. Moreover, preferably, the extent of an observed difference, i.e. an increase or decrease, is preferably, an increase or decrease according to the factor indicated in the Tables, below.

Preferably, the at least one biomarker when selected from Tables 1a, 1c, 2a, 3a, 4a, 5a, 6a, 6c, 7a, 8a, 8c, 9a, 9c or 12a is increased with respect to a reference obtained from a sample derived from a subject or group of subjects which has not been brought into contact with 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate or Thallium(I) acetate or a sample obtained from a healthy subject or group of subjects as indicated in the said Tables.

Preferably, the at least one biomarker when selected from Tables 1b, 1d, 2b, 3b, 4b, 5d, 6d, 6d, 7b, 8b, 8d, 9b, 9d or 12b is decreased with respect to a reference obtained from a sample derived from a subject or group of subjects which has not been brought into contact with 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate or Thallium(I) acetate or a sample obtained from a healthy subject or group of subjects as indicated in the said Tables.

The comparison is, preferably, assisted by automation. For example, a suitable computer program comprising algorithm for the comparison of two different data sets (e.g., data sets comprising the values of the characteristic feature(s)) may be used. Such computer programs and algorithm are well known in the art. Notwithstanding the above, a comparison can also be carried out manually.

The term “substance for treating neuronal toxicity” refers to compounds which may directly interfere with the biological mechanisms inducing neuronal toxicity referred to elsewhere in this specification Alternatively, but also preferred, the compounds may interfere with the development or progression of symptoms associated with the neuronal toxicity. Substances to be identified by the method of the present invention may be organic and inorganic chemicals, such as small molecules, polynucleotides, oligonucleotides including siRNA, ribozymes or micro RNA molecules, peptides, polypeptides including antibodies or other artificial or biological polymers, such as aptameres. Preferably, the substances are suitable as drugs, pro-drugs or lead substances for the development of drugs or pro-drugs. Thus, in an aspect of the invention, the method may further include a step comprising identifying and/or confirming the identified and selected substance a drug, pro-drug or drug or prodrug candidate for further clinical development. Such clinical development may, preferably, includes pharmacological studies of the substance, toxicological determinations of the substance, animal and human drug testing, including clinical trials of all phases.

It is to be understood that if the methods of the present invention are to be used for identifying drugs for the therapy of neuronal toxicity or for toxicological assessments of compounds (i.e. determining whether a compound is capable of inducing neuronal toxicity), test samples of a plurality of subjects may be investigated for statistical reasons. Preferably, the metabolome within such a cohort of test subjects shall be as similar as possible in order to avoid differences which are caused, e.g., by factors other than the compound to be investigated. Subjects to be used for the said methods are, preferably, laboratory animals such as rodents and more preferably rats. It is to be understood further that the said laboratory animals shall be, preferably, sacrificed after completion of the methods of the present invention. All subjects of a cohort test and reference animals shall be kept under identical conditions to avoid any differential environmental influences. Suitable conditions and methods of providing such animals are described in detail in WO2007/014825. Said conditions are hereby incorporated by reference.

Accordingly, the methods of the invention aiming at identifying a substance for treating neuronal toxicity and, in particular, CNS toxicity, eye toxicity and/or peripheral neuronal toxicity including impaired neuromuscular transmission associated with skeletal muscle innervation stimulation, preferably, include additional steps. Preferably, further steps include carrying out preclinical studies with the substance in order to identify pharmacological and/or toxicological parameters thereof, such as ED50/EC50 and/or LD50/LC50 thresholds, carrying out clinical trials, e.g., for determining therapeutic efficacy and safety of the substance and the formulation of the identified substance in a pharmaceutically acceptable form.

The substance can, preferably, be formulated for topical or systemic administration. Conventionally, a drug will be administered intra-muscular or, subcutaneous. However, depending on the nature and the mode of action of a substance, it may, however, be administered by other routes as well. The substance is, preferably, formulated for administration in conventional dosage forms and prepared by combining the identified substance with standard pharmaceutical carriers according to conventional procedures. These procedures may involve mixing, granulating, and compression, or dissolving the ingredients as appropriate to the desired preparation. It will be appreciated that the form and character of the pharmaceutical acceptable carrier or diluent is dictated by the amount of active ingredient with which it is to be combined, the route of administration, and other well-known variables. A carrier must be acceptable in the sense of being compatible with the other ingredients of the formulation and being not deleterious to the recipient thereof. The pharmaceutical carrier employed may include a solid, a gel, or a liquid. Without being limiting, examples for solid carriers are lactose, terra alba, sucrose, talc, gelatin, agar, pectin, acacia, magnesium stearate, stearic acid and the like. Without being limiting, exemplary of liquid carriers are phosphate buffered saline solution, syrup, oil, water, emulsions, various types of wetting agents, and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa. A diluent is selected so as not to affect the biological activity of the combination. Without being limiting, examples of such diluents are distilled water, physiological saline, Ringer's solutions, dextrose solution, and Hank's solution. In addition, the pharmaceutical composition or formulation may also include other carriers, adjuvants, or non-toxic, non-therapeutic, non-immunogenic stabilizers and the like. It is to be understood that the formulation of a substance as a drug takes place under GMP standardized conditions or the like in order to ensure quality, pharmaceutical security, and effectiveness.

The methods of the present invention can be, preferably, implemented by the device of the present invention. A device as used herein shall comprise at least the aforementioned units. The units of the device are operatively linked to each other. How to link the units in an operating manner will depend on the type of units included into the device. For example, where means for automatically qualitatively or quantitatively determining the at least one biomarker are applied in an analyzing unit, the data obtained by said automatically operating unit can be processed by the evaluation unit, e.g., by a computer program which runs on a computer being the data processor in order to facilitate the diagnosis. Preferably, the units are comprised by a single device in such a case. However, the analyzing unit and the evaluation unit may also be physically separate. In such a case operative linkage can be achieved via wire and wireless connections between the units which allow for data transfer. A wireless connection may use Wireless LAN (WLAN) or the internet. Wire connections may be achieved by optical and non-optical cable connections between the units. The cables used for wire connections are, preferably, suitable for high throughput data transport

A preferred analyzing unit for determining at least one biomarker comprises a detection agent, such as an antibody, protein or aptamere which specifically recognizes the at least one biomarker as specified elsewhere herein, and a zone for contacting said detection agent with the sample to be tested. The detection agent may be immobilized on the zone for contacting or may be applied to the said zone after the sample has been loaded. The analyzing unit shall be, preferably, adapted for qualitatively and/or quantitatively determine the amount of complexes of the detection agent and the at least one biomarker. It will be understood that upon binding of the detection agent to the at least one biomarker, at least one measurable physical or chemical property of either the at least one biomarker, the detection agent or both will be altered such that the said alteration can be measured by a detector, preferably, comprised in the analyzing unit. However, where analyzing units such as test stripes are used, the detector and the analyzing units may be separate components which are brought together only for the measurement. Based on the detected alteration in the at least one measurable physical or chemical property, the analyzing unit may calculate an intensity value for the at least one biomarker as specified elsewhere herein. Said intensity value can then be transferred for further processing and evaluation to the evaluation unit. Most preferably, the amount of the at least one biomarker can be determined by ELISA, EIA, or RIA based techniques using a detection agent as specified elsewhere herein. Alternatively, an analyzing unit as referred to herein, preferably, comprises means for separating biomarkers, such as chromatographic devices, and means for biomarker determination, such as spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS and/or GC-MS is used in the analyzing unit referred to in accordance with the present invention.

The evaluation unit of the device of the present invention, preferably, comprises a data processing device or computer which is adapted to execute rules for carrying out the comparison as specified elsewhere herein. Moreover, the evaluation unit, preferably, comprises a database with stored references. A database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for neuronal toxicity (e.g. a query search). Thus, if an identical or similar data set can be identified in the data collection, the test data set will be associated with neuronal toxicity. The evaluation unit may also preferably comprise or be operatively linked to a further database with recommendations for therapeutic or preventive interventions or life style adaptations based on the established diagnosis of neuronal toxicity. Said further database can be, preferably, automatically searched with the diagnostic result obtained by the evaluation unit in order to identify suitable recommendations for the subject from which the test sample has been obtained in order to treat or prevent neuronal toxicity.

In a preferred embodiment of the device of the present invention, said stored reference is a reference derived from a subject or a group of subjects known to suffer from neuronal toxicity or a subject or group of subjects which has been brought into contact with at least one compound selected from the group consisting of 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate or Thallium(I) acetate, and said data processor executes instructions for comparing the amount of the at least one biomarker determined by the analyzing unit to the stored reference, wherein an essentially identical amount of the at least one biomarker in the test sample in comparison to the reference is indicative for the presence of neuronal toxicity or wherein an amount of the at least one biomarker in the test sample which differs in comparison to the reference is indicative for the absence of neuronal toxicity.

In another preferred embodiment of the device of the present invention, said stored reference is a reference derived from a subject or a group of subjects known not to suffer from neuronal toxicity or a subject or group of subjects which has not been brought into contact with at least one compound selected from the group consisting of 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, or Thallium(I) acetate, and said data processor executes instructions for comparing the amount of the at least one biomarker determined by the analyzing unit to the stored reference, wherein an amount of the at least one biomarker in the test sample which differs in comparison to the reference is indicative for the presence of neuronal toxicity or wherein an essentially identical amount of the at least one biomarker in the test sample in comparison to the reference is indicative for the absence of neuronal toxicity.

The device, thus, can also be used without special medical knowledge by medicinal or laboratory staff or patients, in particular when an expert system making recommendations is included. The device is also suitable for near-patient applications since the device can be adapted to a portable format.

The term “kit” refers to a collection of the aforementioned components, preferably, provided separately or within a single container. The container also comprises instructions for carrying out the method of the present invention. These instructions may be in the form of a manual or may be provided by a computer program code which is capable of carrying out the comparisons referred to in the methods of the present invention and to establish a diagnosis accordingly when implemented on a computer or a data processing device. The computer program code may be provided on a data storage medium or device such as an optical or magnetic storage medium (e.g., a Compact Disc (CD), CD-ROM, a hard disk, optical storage media, or a diskette) or directly on a computer or data processing device. A “standard” as referred to in connection with the kit of the invention is an amount of the at least one biomarker when present in solution or dissolved in a predefined volume of a solution resembles the amount of the at least one biomarker which is present (i) in a subject or a group of subjects known to suffer from neuronal toxicity or a subject or group of subjects which has been brought into contact with at least one compound selected from the group consisting of 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, and Thallium(I) acetate or (ii) derived from a subject or a group of subjects known to not suffer from therefrom or a subject or group of subjects which has not been brought into contact with at least one compound selected from the group consisting of 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride Toxaphene, Glipizide, Lead acetate trihydrate, and Thallium(I) acetate.

Advantageously, it has been found in the study underlying the present invention that the amount of at least one biomarker as specified herein allows for diagnosing neuronal toxicity, specifically neuronal toxicity induced by 17-alpha-Ethynylestradiol, Apomorphine, [3-(4,5-dihydro-isoxazol-3-yl)-4-methylsulfonyl-2-chlor phenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride Toxaphene, Glipizide, Lead acetate trihydrate or Thallium(I) acetate. The specificity and accuracy of the method will be even more improved by determining an increasing number or even all of the aforementioned biomarkers. A change in the quantitative and/or qualitative composition of the metabolome with respect to these specific biomarkers is indicative for neuronal toxicity even before other signs of the said toxicity are clinically apparent. The morphological, physiological as well as biochemical parameters which are currently used for diagnosing neuronal toxicity are less specific and less sensitive in comparison to the biomarker determination provided by the present invention. Thanks to the present invention, neuronal toxicity of a compound can be more efficiently and reliably assessed. Moreover, based on the aforementioned findings, screening assays for drugs which are useful for the therapy of neuronal toxicity are feasible. In general, the present invention contemplates the use of at least one biomarker in a sample of a subject selected from any one of the Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9a, 9b, 9c, 9d, 12a or 12b or a detection agent for said biomarker for diagnosing neuronal toxicity, for determining whether a compound is capable of inducing neuronal toxicity or for identifying a substance capable of treating neuronal toxicity. Further, the present invention, in general, contemplates the use of the at least one biomarker in a sample of a subject or a detection agent therefor for identifying a subject being susceptible for a treatment of neuronal toxicity. Preferred detection agents to be used in this context of the invention are those referred to elsewhere herein. Moreover, the methods of the present invention can be, advantageously, implemented into a device. Furthermore, a kit can be provided which allows for carrying out the methods.

The present invention also relates to a data collection comprising characteristic values for the biomarkers recited in any one of Tables 1a, 1b, 1c, 1d, 1e, 1f, 2a, 2b, 3a, 3b, 3c, 3d, 3e, 3f, 3g, 4a, 4b, 4c, 4d, 5a, 5b, 5c, 5d, 6a, 6b, 7a, 7b, 8a, 8b, 9a, 9b, 9c, 9d, 12a, or 12b. The term “data collection” refers to a collection of data which may be physically and/or logically grouped together. Accordingly, the data collection may be implemented in a single data storage medium or in physically separated data storage media being operatively linked to each other. Preferably, the data collection is implemented by means of a database. Thus, a database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for neuronal toxicity (e.g. a query search). Thus, if an identical or similar data set can be identified in the data collection, the test data set will be associated with neuronal toxicity. Consequently, the information obtained from the data collection can be used to diagnose neuronal toxicity based on a test data set obtained from a subject.

Moreover, the present invention pertains to a data storage medium comprising the said data collection. The term “data storage medium” as used herein encompasses data storage media which are based on single physical entities such as a CD, a CD-ROM, a hard disk, optical storage media, or a diskette. Moreover, the term further includes data storage media consisting of physically separated entities which are operatively linked to each other in a manner as to provide the aforementioned data collection, preferably, in a suitable way for a query search.

The present invention also relates to a system comprising

(a) means for comparing characteristic values of at least one biomarker of a sample operatively linked to
(b) the data storage medium of the present invention.

The term “system” as used herein relates to different means which are operatively linked to each other. Said means may be implemented in a single device or may be implemented in physically separated devices which are operatively linked to each other. The means for comparing characteristic values of the biomarker operate, preferably, based on an algorithm for comparison as mentioned before. The data storage medium, preferably, comprises the aforementioned data collection or database, wherein each of the stored data sets being indicative for neuronal toxicity. Thus, the system of the present invention allows identifying whether a test data set is comprised by the data collection stored in the data storage medium. Consequently, the system of the present invention may be applied as a diagnostic means in diagnosing neuronal toxicity. In a preferred embodiment of the system, means for determining characteristic values of biomarkers of a sample are comprised. The term “means for determining characteristic values of biomarkers” preferably relates to the aforementioned devices for the determination of biomarkers such as mass spectrometry devices, ELISA devices, NMR devices or devices for carrying out chemical or biological assays for the analytes.

All references referred to above are herewith incorporated by reference with respect to their entire disclosure content as well as their specific disclosure content explicitly referred to in the above description.

The following Examples are merely for the purposes of illustrating the present invention. They shall not be construed, whatsoever, to limit the scope of the invention in any respect.

### EXAMPLES

### Example

### Biomarkers Associated with Neuronal Toxicity

A group of each 5 male and female rats was dosed once daily with the indicated compounds (see Table 10, below for compounds, applied doses and administration details) over 28 days.

Each dose group in the studies consisted of five rats per sex. Additional groups of each 5 male and female animals served as controls. Before starting the treatment period, animals, which were 62-64 days old when supplied, were acclimatized to the housing and environmental conditions for 7 days. All animals of the animal population were kept under the same constant temperature (20-24±3° C.) and the same constant humidity (30-70%). The animals of the animal population were fed ad libitum. The food to be used was essentially free of chemical or microbial contaminants. Drinking water was also offered ad libitum. Accordingly, the water was free of chemical and microbial contaminants as laid down in the European Drinking Water Directive 98/83/EG. The illumination period was 12 hours light followed by 12 hours darkness (12 hours light, from 6:00 to 18:00, and 12 hours darkness, from 18:00 to 6:00). The studies were performed in an AAALAC-approved laboratory in accordance with the German Animal Welfare Act and the European Council Directive 86/609/EE. The test system was arranged according to the OECD 407 guideline for the testing of chemicals for repeated dose 28-day oral toxicity study in rodents. The test substances (compounds) in the Tables 1 to 9 below were dosed and administered as described in the Table 10 above.

In the morning of day 7, 14, and 28, blood was taken from the retro-orbital venous plexus from fasted anaesthetized animals. From each animal, 1 ml of blood was collected with EDTA as anticoagulant. The samples were centrifuged for generation of plasma. All plasma samples were covered with a N₂ atmosphere and then stored at −80° C. until analysis.

For mass spectrometry-based metabolite profiling analyses plasma samples were extracted and a polar and a non-polar (lipid) fraction was obtained. For GC-MS analysis, the non-polar fraction was treated with methanol under acidic conditions to yield the fatty acid methyl esters. Both fractions were further derivatised with O-methyl-hydroxyamine hydrochloride and pyridine to convert Oxogroups to O-methyloximes and subsequently with a silylating agent before analysis. In LC-MS analysis, both fractions were reconstituted in appropriate solvent mixtures. HPLC was performed by gradient elution on reversed phase separation columns. Mass spectrometric detection which allows target and high sensitivity MRM (Multiple Reaction Monitoring) profiling in parallel to a full screen analysis was applied as described in WO2003073464.

Steroids and their metabolites were measured by online SPE-LC-MS (Solid phase extraction-LC-MS). Catecholamines and their metabolites were measured by online SPE-LC-MS as described by Yamada et al. (Yamada 2002, Journal of Analytical Toxicology, 26(1): 17-22))

Following comprehensive analytical validation steps, the data for each analyte were normalized against data from pool samples. These samples were run in parallel through the whole process to account for process variability. The significance of treatment group values specific for sex, treatment duration and metabolite was determined by comparing means of the treated groups to the means of the respective untreated control groups using WELCH-test and quantified with treatment ratios versus control and p-values.

The identification of the most important biomarkers per toxicity pattern was done by a ranking of the analytes in the tables below. Therefore the metabolic changes in reference treatments of a given pattern (shown in the table) were compared with changes of the same metabolite in other unrelated treatments. For each metabolite T-values were obtained for the reference and control treatment and compared by the Welch test to asses whether these two groups are significantly different. The maximum absolute value of the respective TVALUE was taken to indicate the most important metabolite for the pattern.

The changes of the group of plasma metabolites being indicative for neuronal toxicity after treatment of the rats are shown in the following tables:

TABLE 1a
Markers for CNS GABA receptor agonist in male rats; Significant
up-regulation changes (p-Value ≦ 0.2) are marked (*). For
some metabolites (marked with #), additional information are
provided in table 10. All compounds were administered in low dose.

| | | Pentobarbital | Phenobarbital |
|---|---|---|---|
| | Phenytoin | sodium i.p. | sodium |
| Metabolite | m28 | m28 | m28 |
| Phosphate (inorganic and | 1.25 * | 1.21 * | 1.17 * |
| organic phosphates) | | | |
| 3-Indoxylsulfate | 1.78 | 1.44 * | 1.81 * |
| alpha-Tocopherol | 1.16 * | 1.23 * | 1.14 * |
| Pantothenic acid | 1.25 * | 1.14 | 1.33 * |

TABLE 1b
Markers for CNS GABA receptor agonist in male rats; Significant
down-regulation changes (p-Value ≦ 0.2) are marked (*). For
some metabolites (marked with #), additional information are
provided in table 10. All compounds were administered in low dose.

| | | Pentobarbital | Phenobarbital |
|---|---|---|---|
| | Phenytoin | sodium i.p. | sodium |
| Metabolite | m28 | m28 | m28 |
| Phosphatidylcholine | 0.95 * | 0.94 * | 0.93 * |
| (C16:0, C22_6)# | | | |
| Phosphatidylcholine | 0.99 * | 1 * | 0.99 * |
| (C16:0, C20:4) | | | |
| Cysteine | 0.73 * | 0.64 * | 0.86 * |
| Ketoleucine | 0.76 * | 1.09 | 0.88 * |
| Malate | 0.84 * | 0.83 * | 0.69 * |

TABLE 1c
Markers for CNS GABA receptor agonist in female rats; Significant
up-regulation changes (p-Value ≦ 0.2) are marked (*). For
some metabolites (marked with #), additional information are
provided in table 10. All compounds were administered in low dose.

| | Phenobarbital | |
|---|---|---|
| | sodium | Phenytoin |
| Metabolite | f28 | f28 |
| Coenzyme Q9 | 1.08 * | 1.22 * |
| Isoleucine | 1.12 * | 1.23 * |
| Valine | 1.15 * | 1.36 * |

TABLE 1d
Markers for CNS GABA receptor agonist in female rats; Significant
down-regulation changes (p-Value ≦ 0.2) are marked (*). For
some metabolites (marked with #), additional information are
provided in table 10. All compounds were administered in low dose.

| | Phenobarbital | |
|---|---|---|
| | sodium | Phenytoin |
| Metabolite | f28 | f28 |
| Glutamate | 0.78 * | 0.81 * |
| Cysteine | 0.65 * | 0.86 * |
| myo-Inositol | 0.94 * | 0.69 * |
| Pyruvate | 0.73 * | 0.65 * |

TABLE 2a
Markers for CNS Psycoanaleptics in male rats; Significant up-regulation
changes (p-Value ≦0.2) are marked (*). For some metabolites (marked
with #), additional information are provided in table 10. All compounds
were administered in high dose.

| | Dextroamphetamine | | | Escitalopram | | | | | |
|---|---|---|---|---|---|---|---|---|---|
| | sulfate | | | oxalate | | | Pentoxifylline | | |
| Metabolite | m7 | m14 | m28 | m7 | m14 | m28 | m7 | m14 | m28 |
| Choline plasmalogen | 1.01 | 1.32* | 1.08* | 1.23* | 1.09* | 1.08* | 1.01 | 1.29* | 1.14* |
| No 03# | | | | | | | | | |
| Isoleucine | 1.15 | 1.08* | 1.24* | 1.19* | 1.44* | 1.26* | 0.93 | 1.05* | 1.15* |
| Valine | 1.12 | 1.11* | 1.15* | 1.1 | 1.24* | 1.19* | 0.98* | 1.05* | 1.1* |
| Creatine | 1.34* | 1.16* | 1.33* | 0.97 | 1.27* | 1.19* | 0.95 | 1.36* | 1.18* |

TABLE 2b
Markers for CNS Psycoanaleptics in male rats; Significant down-regulation
changes (p-Value ≦0.2) are marked (*). For some metabolites (marked
with #), additional information are provided in table 10. All compounds
were administered in high dose.

| | Dextroamphetamine | | | Escitalopram | | | | | |
|---|---|---|---|---|---|---|---|---|---|
| | sulfate | | | oxalate | | | Pentoxifylline | | |
| Metabolite | m7 | m14 | m28 | m7 | m14 | m28 | m7 | m14 | m28 |
| DAG | 0.57* | 0.73 | 0.65 | 0.69* | 0.71* | 0.83* | 0.51* | 0.88* | 0.72* |
| (C18:1,C18:2)# | | | | | | | | | |
| Glutamate | 0.93* | 1.06 | 0.88* | 0.77* | 0.76* | 0.71* | 0.74* | 0.84 | 0.79* |
| TAG No 07# | 0.54* | 0.88 | 0.5* | 0.38* | 1.18 | 0.84 | 0.46* | 0.93 | 0.74* |
| Adrenaline (Epinephrine) | 0.54* | 0.16* | 0.48* | 0.3* | 0.32* | 0.16* | 0.34* | 1.34 | 0.33* |
| Metanephrine | 1.06 | 0.55* | 0.42* | 0.39* | 0.36* | 0.41* | 0.48* | 0.61* | 0.63* |
| Glycerol, polar | 0.86* | 0.87* | 0.76* | 1.12 | 0.74* | 0.88* | 0.73* | 0.74* | 0.8 |
| fraction | | | | | | | | | |
| Pyruvate | 0.99 | 0.78* | 0.57* | 0.65* | 0.6* | 0.57* | 0.76* | 0.62* | 0.74* |

TABLE 3a
Markers for CNS Antipsychotic drugs (dopamine receptor block)
in male rats; Significant up-regulation changes (p-Value ≦0.1)
are marked (*). For some metabolites (marked with #),
additional information are provided in table 10. All compounds
were administered in high dose.

| | | | | Ziprasidone | | |
|---|---|---|---|---|---|---|
| | Risperidone | | | hydrochloride | | |
| Metabolite | m7 | m14 | m28 | m7 | m14 | m28 |
| myo-Inositol-2-phosphate, | 1.34 | 1.71* | 1.28* | 1.41* | 1.44* | 1.63* |
| lipid fraction | | | | | | |
| Tryptophan | 1.23* | 1.09* | 1.24* | 1.1 | 1.16* | 1.18* |
| 3-Methoxytyrosine | 0.99 | 1.06* | 1.57* | 1.12 | 1.33* | 1.47* |
| Arginine | 1.18* | 1.25* | 1.13 | 1.22* | 1.11* | 0.98 |

TABLE 3b
Markers for CNS Antipsychotic drugs (dopamine receptor block)
in male rats; Significant down-regulation changes (p-Value ≦0.1)
are marked (*). For some metabolites (marked with #), additional
information are provided in table 10. All compounds were
administered in high dose.

| | | | | Ziprasidone | | |
|---|---|---|---|---|---|---|
| | Risperidone | | | hydrochloride | | |
| Metabolite | m7 | m14 | m28 | m7 | m14 | m28 |
| Threonic acid | 0.84* | 0.76* | 0.55* | 0.66* | 0.52* | 0.63* |
| Glycine | 0.92 | 0.86* | 0.78* | 0.82* | 0.95 | 0.86* |

TABLE 4a
Markers for CNS dopamine antagonist in female rats; Significant
up-regulation changes (p-Value ≦0.1) are marked (*). For some
metabolites (marked with #), additional information are provided
in table 10. All compounds were administered in high dose.

| | | | | Ziprasidone | | | | | | | | |
|---|---|---|---|---|---|---|---|---|---|---|---|---|
| | Risperidone | | | hydrochloride | | | Chlorpromazine | | | Olanzapine | | |
| Metabolite | f7 | f14 | f28 | f7 | f14 | f28 | f7 | f14 | f28 | f7 | f14 | f28 |
| Phosphatidylcholine | 1.13* | 1.2* | 1.16* | 1.4* | 1.32* | 1.32* | 1.11* | 1.3* | 1.14 | 1.15* | 1.14* | 1.23* |
| (C16:0,C16:0)# | | | | | | | | | | | | |
| Docosapentaenoic acid | 4.1* | 3.68* | 2.78* | 2.26* | 1.55* | 1.54* | 1.46 | 1.17 | 1.18* | 2.64* | 2.83* | 2.39* |
| (C22:cis[7,10,13,16,19]5) | | | | | | | | | | | | |
| Phosphatidylcholine | 1.54* | 1.6* | 1.61* | 1.41* | 1.06 | 1.5* | 1.38* | 1.02 | 1 | 1.3* | 1.39* | 1.4* |
| (C16:1,C18:2)# | | | | | | | | | | | | |
| Ceramide (d18:1,C24:1) | 1.59* | 1.67* | 1.07* | 1.23 | 1.47* | 1.2 | 2.34* | 1.44* | 1.22 | 1.79* | 1.03 | 1.41* |
| myo-Inositol, lipid fraction | 1.48* | 1.38* | 1.37* | 1.41* | 1.26* | 1.2 | 1.42 | 1.02 | 0.96 | 1.63* | 1.36* | 1.47* |
| Elaidic acid | 1.5* | 1.41* | 1.12* | 1.52* | 0.97 | 1.18 | 1.46 | 1.16* | 0.98 | 1.56* | 1.35* | 1.28* |
| (C18:trans[9]1) | | | | | | | | | | | | |
| Progesterone | 10.16* | 0.94 | 0.93 | 9.81* | 1.28 | 2.41* | NA | NA | NA | 12.82* | 1.33 | 5.79* |
| Galactose, lipid fraction | 1.51* | 1.5* | 1.38* | 1.46* | 1.17* | 1.22 | 1.34* | 1 | 0.91 | 1.41* | 1.24 | 1.38* |
| TAG No 07# | 5.76* | 6.65* | 3.47* | 3.4* | 0.62 | 1.02 | 2.57* | 1.79 | 2.1* | 2.45* | 2.34* | 2.59* |
| Myristic acid (C14:0) | 2.08* | 1.9* | 2.05* | 1.53* | 1.09 | 1.18 | 0.91 | 1.04 | 0.89 | 1.6 | 1.51* | 1.66* |

TABLE 4b
Markers for CNS dopamine antagonists in female rats; Significant
down-regulation changes (p-Value ≦0.1) are marked (*). For some
metabolites (marked with #), additional information are provided
in table 10. All compounds were administered in high dose.

| | | | | Ziprasidone | | | | | | | | |
|---|---|---|---|---|---|---|---|---|---|---|---|---|
| | Risperidone | | | hydrochloride | | | Chlorpromazine | | | Olanzapine | | |
| Metabolite | f7 | f14 | f28 | f7 | f14 | f28 | f7 | f14 | f28 | f7 | f14 | f28 |
| Creatinine | 0.79 | 0.7 | 0.91* | 0.87 | 1.02 | 0.89* | 0.97 | 0.86 | 0.84* | 0.9 | 0.93 | 0.63* |
| Serotonin (5-HT) | 0.16* | 0.44 | 0.18* | 0.11* | 0.59 | 1.37 | NA | NA | NA | 0.22* | 0.3* | 1.03 |
| Uric acid | 0.78* | 0.42 | 0.42* | 0.51* | 0.5* | 0.73* | 0.62* | 0.84 | 0.88 | 0.64* | 0.64* | 0.97 |
| Glycine | 0.98 | 0.83* | 0.82 | 0.86* | 0.86* | 0.91 | 1.01 | 1.06* | 1.01 | 0.82* | 0.68* | 0.76 |
| Myristic acid | 2.08 | 1.9 | 2.05 | 1.53 | 1.09 | 1.18 | 0.91 | 1.04 | 0.89* | 1.6 | 1.51 | 1.66 |
| (C14:0) | | | | | | | | | | | | |
| Normetanephrine | 0.67* | 0.59* | 0.73* | 0.73* | 0.88 | 0.95 | NA | NA | NA | 1.02 | 0.77* | 1.01 |
| Lactate | 0.66* | 0.86 | 0.74* | 0.6* | 0.66 | 0.62* | 0.65* | 0.97 | 0.94 | 0.6 | 0.88 | 0.72* |
| Phosphocreatine | NA | NA | NA | 0.61* | NA | 0.47 | NA | NA | NA | NA | NA | NA |
| Glutamate | 1.03 | 0.77* | 0.89 | 0.67* | 0.71* | 0.76* | 0.83* | 0.93* | 1.05 | 0.69* | 0.73* | 0.71* |

TABLE 5a
Markers for CNS receptor block (Serotonine-Dopamine) in female
rats; Significant up-regulation changes (p-Value ≦0.1) are marked
(*). For some metabolites (marked with #), additional information
are provided in table 10. All compounds were administered in high dose.

| | | | | Escitalopram | | | Quetiapine | | |
|---|---|---|---|---|---|---|---|---|---|
| | Risperidone | | | oxalate | | | fumarate | | |
| Metabolite | f7 | f14 | f28 | f7 | f14 | f28 | f7 | f14 | f28 |
| Glutamine | 1.4* | 1.34* | 1.2 | 1.35* | 1.27* | 1.21 | 1.29* | 1.17* | 1.31* |
| Galactose, lipid | 1.51* | 1.5* | 1.38* | 1.14* | 1.27* | 1.23* | 1.18* | 1.16* | 1.21 |
| fraction | | | | | | | | | |
| Valine | 1.11* | 1.07 | 1.1* | 1.37* | 1.28* | 1.15* | 1.49* | 1.24* | 1.12* |
| Leucine | 1.13 | 1.1* | 1.18* | 1.54* | 1.48* | 1.32* | 1.98* | 1.44* | 1.27* |
| Isoleucine | 1.19* | 1.03 | 1.14* | 1.47* | 1.39* | 1.28* | 1.9* | 1.46* | 1.3* |
| Histidine | 1.2* | 1.3* | 1.25 | 1.24* | 1.16 | 1.11* | 1.31* | 1.15* | 1.06* |
| 3-Hydroxybutyrate | 1.15* | 1.06 | 1.31* | 1.51* | 1.32 | 1.23 | 1.49* | 1.55* | 2.04* |

TABLE 5b
Markers for CNS receptor block (Serotonine-Dopamine) in female
rats; Significant down-regulation changes (p-Value ≦0.1) are
marked (*). For some metabolites (marked with #), additional
information are provided in table 10. All compounds were
administered in high dose.

| | | | | Escitalopram | | | Quetiapine | | |
|---|---|---|---|---|---|---|---|---|---|
| | Risperidone | | | oxalate | | | fumarate | | |
| Metabolite | f7 | f14 | f28 | f7 | f14 | f28 | f7 | f14 | f28 |
| Pyruvate | 0.58* | 0.93 | 0.51* | 0.31* | 0.26* | 0.29* | 0.37* | 0.33* | 0.36* |
| 4-Hydroxy-3- | 0.86* | 0.74* | 0.76* | 0.57* | 0.61* | 0.69* | 0.73* | 0.67* | 0.8 |
| methoxyphenylglycol | | | | | | | | | |
| (HMPG) | | | | | | | | | |
| Lactate | 0.66* | 0.86 | 0.74* | 0.39* | 0.48* | 0.42* | 0.45* | 0.61* | 0.46* |
| Metanephrine | 1.15 | 0.86* | 0.79* | 0.44* | 0.24* | 0.31* | 0.55* | 0.26* | 0.38* |
| Uric acid | 0.78* | 0.42 | 0.42* | 0.78* | 0.78 | 0.86 | 0.92 | 0.64* | 0.68* |
| Glucose | 0.94 | 0.89* | 0.88* | 0.93 | 0.87* | 0.84* | 0.94 | 0.87* | 0.85* |
| Malate | 0.79* | 1.01 | 0.68* | 0.51* | 0.75* | 0.52* | 0.67* | 0.66* | 0.55* |

TABLE 6a
Markers for Nerve System dopamin agonist in female rats; Significant up-regulation changes
(p-Value ≦0.2) are marked (*). For some metabolites (marked with #), additional
information are provided in table 10. All compounds were administered in high dose.

| | Bromocriptine | | Raloxifene | Selegiline | 17-alpha- | |
|---|---|---|---|---|---|---|
| | Mesylate | Cabergoline | Hydrochloride | hydrochloride | Ethynylestradiol | Apomorphine |
| Metabolite | f28 | f28 | f28 | f28 | f28 | f28 |
| 3- | NA | 1.24* | 1.09 | 1.38* | 1.29* | NA |
| Methoxytyrosine | | | | | | |
| Isoleucine | 1.14* | 1.14* | 1.25* | 1.18* | 1.07 | 1.25* |

TABLE 6b
Markers for Nerve System dopamin agonist in female rats; Significant down-regulation changes
(p-Value ≦0.2) are marked (*). For some metabolites (marked with #), additional
information are provided in table 10. All compounds were administered in high dose.

| | Bromocriptine | | Raloxifene | Selegiline | 17-alpha- | |
|---|---|---|---|---|---|---|
| | Mesylate | Cabergoline | Hydrochloride | hydrochloride | Ethynylestradiol | Apomorphine |
| Metabolite | f28 | f28 | f28 | f28 | f28 | f28 |
| Ceramide | 0.6* | 0.39* | 0.71 | 0.48* | 0.6* | 0.61* |
| (d18:1,C24:1) | | | | | | |
| 16- | 0.51* | 0.28* | 0.63* | 0.55* | 0.29* | 0.66* |
| Methylheptadecanoic | | | | | | |
| acid | | | | | | |
| Homovanillic acid | NA | 0.41* | 0.58* | 0.45* | 0.47* | NA |
| (HVA) | | | | | | |
| Choline plasmalogen | 0.62* | 0.39* | 0.75* | 0.82 | 0.38* | 0.89* |
| No 03# | | | | | | |
| 17- | 0.67* | 0.3* | 0.5* | 0.57* | 0.46* | 0.73* |
| Methyloctadecanoic | | | | | | |
| acid | | | | | | |
| Phosphatidylcholine | 0.47* | 0.34* | 0.53* | 0.84 | 0.39* | 0.88* |
| No 04# | | | | | | |
| Pyruvate | 0.71* | 0.93 | 0.45* | 0.36* | 0.87* | 0.33* |
| Phosphatidylcholine | 0.94* | 0.97 | 1 | 0.94* | 0.78* | 0.88* |
| (C16:0,C20:4) | | | | | | |
| 4-Hydroxy-3- | NA | 0.79* | 0.6* | 0.3* | 0.7* | NA |
| methoxyphenylglycol | | | | | | |
| (HMPG) | | | | | | |
| 14- | 0.85* | 0.74* | 0.89 | 1.04 | 0.86* | 0.83* |
| Methylhexadecanoic | | | | | | |
| acid | | | | | | |
| Myristic acid (C14:0) | 0.84 | 0.33* | 0.9 | 0.97 | 0.59* | 0.9* |
| Isopalmitic acid | 0.62* | 0.36* | 0.82 | 0.69* | 0.51* | 0.93 |
| (C16:0) | | | | | | |
| Lactate | 0.47* | 0.7* | 0.64* | 0.72* | 0.99 | 0.9 |

TABLE 6c
Markers for Nerve system serotonin receptor antagonist in female rats; Significant up-regulation changes
(p-Value ≦0.2) are marked (*). For some metabolites (marked with #), additional information are
provided in table 10. All compounds were administered in high dose.

| | | | | Ziprasidone | | | | | | Quetiapine | | |
|---|---|---|---|---|---|---|---|---|---|---|---|---|
| | Risperidone | | | hydrochloride | | | Olanzapine | | | fumarate | | |
| Metabolite | f7 | f14 | f28 | f7 | f14 | f28 | f7 | f14 | f28 | f7 | f14 | f28 |
| Docosapentaenoic acid | 4.1* | 3.68* | 2.78* | 2.26* | 1.55* | 1.54* | 2.64* | 2.83* | 2.39* | 1.98* | 2.01* | 1.76* |
| (C22:cis[7,10,13,16,19]5) | | | | | | | | | | | | |
| Phosphatidylcholine | 1.13* | 1.2* | 1.16* | 1.4* | 1.32* | 1.32* | 1.15* | 1.14* | 1.23* | 1.11* | 1.2* | 1.07 |
| (C16:0,C16:0)# | | | | | | | | | | | | |
| Phosphatidylcholine | 1.07* | 1.05* | 1.06* | 1.02 | 1.22* | 1.15* | 1.12* | 1.06* | 1.04* | 1.09* | 1.08* | 1.07* |
| (C16:0,C22:6) | | | | | | | | | | | | |
| Docosahexaenoic acid | 2.52* | 2.14* | 2.02* | 1.7* | 1.43* | 1.26 | 1.72* | 1.41* | 1.55* | 1.67* | 1.58* | 1.24* |
| (C22:cis[4,7,10,13,16,19]6) | | | | | | | | | | | | |
| Phosphatidylcholine No | 1.5* | 1.45* | 1.55* | 1.17* | 1.19* | 1.2* | 1.43* | 1.37* | 1.05 | 1.05 | 1.17* | 1.31* |
| 02# | | | | | | | | | | | | |
| Galactose, lipid fraction | 1.51* | 1.5* | 1.38* | 1.46* | 1.17* | 1.22 | 1.41* | 1.24* | 1.38* | 1.18* | 1.16* | 1.21 |
| Phosphatidylcholine | 1.54* | 1.6* | 1.61* | 1.41* | 1.06 | 1.5* | 1.3* | 1.39* | 1.4* | 1.28* | 1.23* | 1.07 |
| (C16:1,C18:2)# | | | | | | | | | | | | |
| Palmitic acid (C16:0) | 1.94* | 1.95* | 1.69* | 1.56* | 1.12* | 1.2* | 1.64* | 1.73* | 1.64* | 1.28* | 1.24* | 1.32* |
| TAG (C16:0,C16:1)# | 5.33* | 9.21* | 4.05* | 4.38* | 0.81 | 1.75* | 3.07* | 3.02* | 4.13* | 2.83* | 2.16* | 2.14* |
| 4-Hydroxysphinganine | 1.63* | 1.64* | 1.1 | 1.49* | 1.43* | 1.16* | 1.53* | 1.43* | 2.05* | 1.03 | 1.46* | 1.12 |
| (t18:0, Phytosphingosine), | | | | | | | | | | | | |
| total | | | | | | | | | | | | |
| TAG (C16:0,C18:2)# | 3* | 3.04* | 2.23* | 2.47* | 0.91 | 1.38* | 3.26* | 1.92* | 2.45* | 2.65* | 1.82* | 1.84* |
| Phosphatidylcholine | 1.04* | 1.05* | 1.07* | 1.05* | 0.99 | 1.17* | 1.04* | 1.1* | 1.06* | 1.09* | 1.08* | 1.1* |
| (C16:0,C20:5)# | | | | | | | | | | | | |
| TAG | 5.42* | 3.14* | 2.91* | 2.8* | 0.76 | 1.3* | 2.48* | 2.33* | 3.03* | 1.41* | 1.52* | 2.06* |
| (C16:0,C18:1,C18:3)# | | | | | | | | | | | | |
| TAG (C18:2,C18:3)# | 5.67* | 4.97* | 2.97* | 3.48* | 0.76 | 1.46* | 3.95* | 2.36* | 3.76* | 1.83* | 1.37* | 1.93* |
| TAG (C18:2,C18:2)# | 4.31* | 4.14* | 2.33* | 2.44* | 0.81 | 1.41* | 3.21* | 2.3* | 2.7* | 1.99* | 1.5* | 1.41* |
| TAG | 2.3* | 3.51* | 1.79* | 3.89* | 0.91 | 1.44* | 1.44* | 1.7* | 2* | 2.04* | 1.63* | 1.27* |
| (C18:3,C18:2,C18:1)# | | | | | | | | | | | | |
| TAG No 059# | 1.95* | 2.24* | 1.41* | 3.12* | 0.84 | 1.58* | 2.36* | 1.65* | 2.04* | 2.1* | 2.21* | 1.48* |

TABLE 6d
Markers for Nerve system serotonin receptor antagonist in female rats; Significant down-regulation
changes (p-Value ≦0.2) are marked (*). For some metabolites (marked with #), additional
information are provided in table 10. All compounds were administered in high dose.

| | | | | Ziprasidone | | | | | | Quetiapine | | |
|---|---|---|---|---|---|---|---|---|---|---|---|---|
| | Risperidone | | | hydrochloride | | | Olanzapine | | | fumarate | | |
| Metabolite | f7 | f14 | f28 | f7 | f14 | f28 | f7 | f14 | f28 | f7 | f14 | f28 |
| Glycine | 0.98 | 0.83* | 0.82* | 0.86* | 0.86* | 0.91 | 0.82* | 0.68* | 0.76* | 0.82* | 0.71* | 0.86* |
| 4- | 0.24* | 0.27* | 0.37* | 0.42* | 0.21* | 1.02 | 0.54* | 0.65* | 0.87 | 0.61* | 0.62* | 0.36* |
| Hydroxyphenylpyruvate | | | | | | | | | | | | |
| Uric acid | 0.78* | 0.42 | 0.42* | 0.51* | 0.5* | 0.73* | 0.64* | 0.64* | 0.97 | 0.92 | 0.64* | 0.68* |
| Lactate | 0.66* | 0.86 | 0.74* | 0.6* | 0.66 | 0.62* | 0.6* | 0.88 | 0.72* | 0.45* | 0.61* | 0.46* |
| Lysophosphatidylcholine | 0.93* | 0.98* | 0.99* | 0.99* | 1 | 0.98* | 0.97* | 0.98* | 0.98* | 0.97* | 0.98* | 0.97* |
| (C16:0) | | | | | | | | | | | | |

TABLE 7a
Markers for Nerve system serotonin reuptake inhibition in
female rats; Significant up-regulation changes (p-Value ≦0.2)
are marked (*). For some metabolites (marked with #),
additional information are provided in table 10. All
compounds were administered in high dose.

| | Citalopram | | | Paroxetine | | |
|---|---|---|---|---|---|---|
| | hydrobromide | | | hydrochloride | | |
| Metabolite | f7 | f14 | f28 | f7 | f14 | f28 |
| 4- | 1.87* | 1.71* | 1.92* | 1.99* | 1.9* | 2.11* |
| Hydroxysphinganine | | | | | | |
| (t18:0, Phytosphingosine), | | | | | | |
| total | | | | | | |
| Threonine | 1.45* | 1.47* | 1.78* | 1.41* | 1.65* | 1.4* |
| alpha-Tocopherol | 2.13* | 2.08* | 1.78* | 1.08 | 1.31* | 1.32* |

TABLE 7b
Markers for Nerve system serotonin reuptake inhibition in
female rats; Significant down-regulation changes (p-Value ≦0.2)
are marked (*). For some metabolites (marked with #),
additional information are provided in table 10. All
compounds were administered in high dose.

| | Citalopram | | | Paroxetine | | |
|---|---|---|---|---|---|---|
| | hydrobromide | | | hydrochloride | | |
| Metabolite | f7 | f14 | f28 | f7 | f14 | f28 |
| Serotonin (5-HT) | 0.06* | 0.08* | 0.03* | 0.09* | 0.07* | 0.04* |
| Adrenaline (Epi-nephrine) | 0.71 | 0.11* | 0.3* | 0.19* | 0.36* | 0.89 |
| Glutamate | 0.45* | 0.49* | 0.68* | 0.7* | 0.68* | 0.85 |

TABLE 8a
Markers for Nerve System serotonine reuptake inhibition in male rats; Significant up-regulation
changes (p-Value ≦0.2) are marked (*). For some metabolites (marked with #), additional
information are provided in table 10. All compounds were administered in high dose.

| | Citalopram | | | Escitalopram | | | Fluoxetine | | | Sertraline | | |
|---|---|---|---|---|---|---|---|---|---|---|---|---|
| | hydrobromide | | | oxalate | | | hydrochloride | | | hydrochloride | | |
| Metabolite | m7 | m14 | m28 | m7 | m14 | m28 | m7 | m14 | m28 | m7 | m14 | m28 |
| 4- | 1.61* | 1.79* | 1.67* | 1.53* | 1.25 | 1.42* | 1.34* | 1.51* | 2.57* | 1.45* | 1.79* | 1.29 |
| Hydroxysphinganine | | | | | | | | | | | | |
| (t18:0, Phytosphingosine), | | | | | | | | | | | | |
| total | | | | | | | | | | | | |
| Ceramide | 1.38* | 1.47* | 1.72* | 1.29 | 1.45* | 1.39* | 1.77 | 1.91* | 1.85* | 1.68* | 1.46* | 1.49* |
| (d18:1,C24:1) | | | | | | | | | | | | |
| threo-Sphingosine | 1.97* | 1.77* | 1.76* | 1.43* | 1.15 | 1.46* | 1.2 | 1.45* | 2.2* | 1.79* | 1.67* | 1.23 |

TABLE 8b
Markers for Nerve System serotonine reuptake inhibition in male rats;
Significant down-regulation changes (p-Value ≦0.2) are marked (*).
For some metabolites (marked with #), additional information are
provided in table 10. All compounds were administered in high dose.

| | Citalopram | | | Escitalopram | | | Fluoxetine | | | Sertraline | | |
|---|---|---|---|---|---|---|---|---|---|---|---|---|
| | hydrobromide | | | oxalate | | | hydrochloride | | | hydrochloride | | |
| Metabolite | m7 | m14 | m28 | m7 | m14 | m28 | m7 | m14 | m28 | m7 | m14 | m28 |
| DAG | 0.64* | 0.75* | 0.7* | 0.69* | 0.71* | 0.83 | 0.65* | 0.64* | 0.63* | 0.63* | 0.51* | 0.49* |
| (C18:1,C18:2)# | | | | | | | | | | | | |
| Serotonin (5-HT) | 0.09* | 0.1* | 0.2* | 0.09* | 0.09* | 0.2* | 0.33* | 0.07* | 0.28* | 0.26* | 0.26* | 1.29 |

TABLE 8c
Markers for Nervous System dopamin agonist in female rats; Significant
up-regulation changes (p-Value ≦ 0.2) are marked (*). For some
metabolites (marked with #), additional information are provided
in table 10. All compounds were administered in high dose.

| | Apo- | Selegiline | Bromocriptine |
|---|---|---|---|
| | morphine | hydrochloride | Mesylate |
| Metabolite | f28 | f28 | f28 |
| TAG | 2.31 * | 1.81 * | 1.2 * |
| (C16:0, C18:2)# | | | |
| 3-Hydroxybutyrate | 1.41 * | 1.29 * | 1.13 |
| Isoleucine | 1.25 * | 1.18 * | 1.14 * |

TABLE 8d
Markers for Nervous System dopamin agonist in female rats; Significant
down-regulation changes (p-Value ≦ 0.2) are marked (*). For
some metabolites (marked with #), additional information are
provided in table 10. All compounds were administered in high dose.

| | Apo- | Selegiline | Bromocriptine |
|---|---|---|---|
| | morphine | hydrochloride | Mesylate |
| Metabolite | f28 | f28 | f28 |
| 16-Methylheptadecanoic | 0.66 * | 0.55 * | 0.51 * |
| acid | | | |
| Ceramide (d18:1, C24:1) | 0.61 * | 0.48 * | 0.6 * |
| Phosphatidylcholine | 0.88 * | 0.94 * | 0.94 * |
| (C16:0, C20:4) | | | |
| Pyruvate | 0.33 * | 0.36 * | 0.71 * |
| 17-Methyloctadecanoic | 0.73 * | 0.57 * | 0.67 * |
| acid | | | |
| Phosphatidylcholine | 0.98 * | 0.92 * | 0.71 * |
| (C18:0, C20:3)# | | | |

TABLE 9a
Markers for Eye HPPD Inhibition in female rats; Significant
up-regulation changes (p-Value ≦0.1) are marked (*). For
some metabolites (marked with #), additional information
are provided in table 10. All compounds were administered in
high dose.

| | | | | [3-(4,5-dihydro- | | |
|---|---|---|---|---|---|---|
| | | | | isoxazol-3-yl)- | | |
| | | | | 4-methylsulfonyl-2- | | |
| | | | | chlorphenyl](5-hydroxy- | | |
| | NTBC (HPPD- | | | 1-methyl-1H-pyrazol- | | |
| | Inhibitor) | | | 4-yl)methanone | | |
| Metabolite | f7 | f14 | f28 | f7 | f14 | f28 |
| 4-Hydroxy- | 245.75* | 293.51* | 276.92* | 26.68* | 25.92* | 24.95* |
| phenyl- | | | | | | |
| pyruvate | | | | | | |
| Tyrosine | 37.75* | 50.64* | 38.31* | 16.36* | 16.29* | 12.88* |
| Glycine | 1.2* | 1.33* | 1.48* | 1.37* | 1.24* | 1.25* |
| gamma- | 1.34 | 1.61* | 1.55* | 1.44* | 1.55* | 1.77* |
| Linolenic | | | | | | |
| acid | | | | | | |
| (C18:cis[6,9, | | | | | | |
| 12]3) | | | | | | |
| Phosphatidyl- | 1.03* | 1 | 1.05* | 1.12* | 1.12* | 1.11* |
| choline | | | | | | |
| (C18:0,C18:2)# | | | | | | |
| Serine | 1.38* | 1.48* | 1.43* | 1.16* | 1.18* | 1.14* |
| TAG (C18:1, | 2.45* | 2.22* | 1.51* | 2.72* | 2.89* | 3.2* |
| C18:2)# | | | | | | |
| TAG (C16:0, | 1.62* | 1.37* | 1.44* | 2.56* | 2.2* | 2.44* |
| C18:2)# | | | | | | |
| TAG (C18:2, | 1.91* | 1.92* | 1.31* | 3.93* | 2.47* | 2.77* |
| C18:2)# | | | | | | |
| Threonine | 1.43* | 1.82* | 1.52* | 1.16 | 1.33* | 1.24* |

TABLE 9b
Markers for Eye HPPD Inhibition in female rats; Significant
down-regulation changes (p-Value ≦0.1) are marked (*).
For some metabolites (marked with #), additional
information are provided in table 10. All compounds were
administered in high dose.

| | | | | [3-(4,5-dihydro- | | |
|---|---|---|---|---|---|---|
| | | | | isoxazol-3-yl)- | | |
| | | | | 4-methylsulfonyl- | | |
| | | | | 2-chlorphenyl]- | | |
| | | | | (5-hydroxy-1-methyl- | | |
| | NTBC (HPPD- | | | 1H-pyrazol- | | |
| | Inhibitor) | | | 4-yl)methanone | | |
| Metabolite | f7 | f14 | f28 | f7 | f14 | f28 |
| Glutamine | 0.54* | 0.46* | 0.48* | 0.49* | 0.53* | 0.52* |
| 5-Oxoproline | 0.5* | 0.55* | 0.69* | 0.56* | 0.68* | 0.58* |

TABLE 9c
Markers for Eye HPPD Inhibition in male rats; Significant
up-regulation changes (p-Value ≦0.1) are marked (*).
For some metabolites (marked with #), additional
information are provided in table 10. All compounds were
administered in high dose.

| | | | | [3-(4,5-dihydro- | | |
|---|---|---|---|---|---|---|
| | | | | isoxazol-3-yl)- | | |
| | | | | 4-methylsulfonyl- | | |
| | | | | 2-chlorphenyl]- | | |
| | | | | (5-hydroxy-1- | | |
| | NTBC (HPPD- | | | methyl-1H-pyrazol- | | |
| | Inhibitor) | | | 4-yl)methanone | | |
| Metabolite | m7 | m14 | m28 | m7 | m14 | m28 |
| Tyrosine | 56.14* | 58.08* | 52.56* | 21.56* | 18.22* | 14.42* |
| Threonine | 1.36* | 1.51* | 1.54* | 1.16* | 1.2* | 1.5* |
| Lysine | 1.12 | 1.4* | 1.46* | 1.23* | 1.32* | 1.29* |
| 4- | 364.14* | 435.77* | 366.39* | 31.22* | 34.78* | 25.05* |
| Hydroxy- | | | | | | |
| phenylpyruvate | | | | | | |

TABLE 9d
Markers for Eye HPPD Inhibition in male rats; Significant
down-regulation changes (p-Value ≦0.1) are marked (*).
For some metabolites (marked with #), additional
information are provided in table 10. All compounds were
administered in high dose.

| | | | | [3-(4,5-dihydro- | | |
|---|---|---|---|---|---|---|
| | | | | isoxazol-3-yl)- | | |
| | | | | 4-methylsulfonyl- | | |
| | | | | 2-chlorphenyl]- | | |
| | | | | (5-hydroxy-1- | | |
| | NTBC (HPPD- | | | methyl-1H-pyrazol- | | |
| | Inhibitor) | | | 4-yl)methanone | | |
| Metabolite | m7 | m14 | m28 | m7 | m14 | m28 |
| Glutamine | 0.55* | 0.57* | 0.58* | 0.41* | 0.43* | 0.41* |
| Citrulline | 0.84* | 0.88* | 0.83* | 0.73* | 0.72* | 0.8* |
| 5-Oxoproline | 0.6* | 0.61* | 0.6* | 0.5* | 0.58* | 0.54* |
| Phenylalanine | 0.78* | 0.77* | 1.02 | 0.79* | 0.9* | 0.82 |

TABLE 10
Chemical/physical properties of selected analytes. These biomarkers
are characterized herein by chemical and physical properties.

| Metabolite | Fragmentation pattern (GC-MS) and description |
|---|---|
| 3-O-Methylsphingosine (d18:1) | 3-O-Methylsphingosine (d18:1) exhibits the following |
| | characteristic ionic fragments when detected with |
| | GC/MS, applying electron impact (EI) ionization |
| | mass spectrometry, after acidic methanolysis and |
| | derivatisation with 2% O-methylhydroxylamine- |
| | hydrochlorid in pyridine and subsequently with N- |
| | methyl-N-trimethylsilyltrifluoracetamid: MS (EI, 70 |
| | eV): m/z (%): 204 (100), 73 (18), 205 (16), 206 (7), |
| | 354 (4), 442 (1). |
| 5-O-Methylsphingosine (d18:1) | 5-O-Methylsphingosine (d18:1) exhibits the following |
| | characteristic ionic fragments when detected with |
| | GC/MS, applying electron impact (EI) ionization |
| | mass spectrometry, after acidic methanolysis and |
| | derivatisation with 2% O-methylhydroxylamine- |
| | hydrochlorid in pyridine and subsequently with N- |
| | methyl-N-trimethylsilyltrifluoracetamid: MS (EI, 70 |
| | eV): m/z (%): 250 (100), 73 (34), 251 (19), 354 (14), |
| | 355 (4), 442 (1). |
| Cholesterolester No 01 | Metabolite belongs to the class of cholesterolesters. |
| | It exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 369.2 (+/−0.5). |
| Choline plasmalogen No 01 | Metabolite belongs to the class of choline plasmalogens. |
| | It exhibits the following characteristic ionic |
| | species when detected with LC/MS, applying electro- |
| | spray ionization (ESI) mass spectrometry: mass-to- |
| | charge ratio (m/z) of the positively charged ionic |
| | species is 772.6 (+/−0.5). |
| Choline plasmalogen No 02 | Metabolite belongs to the class of choline plasmalogens. |
| | It exhibits the following characteristic ionic |
| | species when detected with LC/MS, applying electro- |
| | spray ionization (ESI) mass spectrometry: mass-to- |
| | charge ratio (m/z) of the positively charged ionic |
| | species is 767 (+/−0.5). |
| Choline plasmalogen No 03 | Metabolite belongs to the class of choline plasmalogens. |
| | It exhibits the following characteristic ionic |
| | species when detected with LC/MS, applying electro- |
| | spray ionization (ESI) mass spectrometry: mass-to- |
| | charge ratio (m/z) of the positively charged ionic |
| | species is 768.8 (+/−0.5). |
| DAG (C18:1, C18:2) | DAG (C18:1, C18:2) represents the sum parameter of |
| | diacylglycerols containing the combination of a C18:1 |
| | fatty acid unit and a C18:2 fatty acid unit. The mass- |
| | to-charge ratio (m/z) of the ionised species is 641.6 |
| | Da (+/−0.5 Da). |
| Eicosaenoic acid (C20:1) | Eicosaenoic acid (C20:1) exhibits the following |
| No 02 | characteristic ionic fragments when detected with |
| | GC/MS, applying electron impact (EI) ionization |
| | mass spectrometry, after acidic methanolysis and |
| | derivatisation with 2% O-methylhydroxylamine- |
| | hydrochlorid in pyridine and subsequently with N- |
| | methyl-N-trimethylsilyltrifluoracetamid: MS (EI, 70 |
| | eV): m/z (%): 55 (100), 69 (75), 41 (57), 83 (54), 74 |
| | (53), 97 (45), 110 (20), 292 (13), 293 (13), 124 (12), |
| | 250 (9), 152 (8), 138 (8), 208 (7), 324 (2). |
| Glycerol phosphate, lipid | Glycerol phosphate, lipid fraction represents the sum |
| fraction | parameter of metabolites containing a glycerol-2- |
| | phosphate or a glycerol-3-phosphate moiety and |
| | being present in the lipid fraction after extraction and |
| | separation of the extract into a polar and a lipid fraction. |
| Lysophosphatidylcholine | Lysophosphatidylcholine (C17:0) represents the sum |
| (C17:0) | parameter of lysoglycerophosphorylcholines containing |
| | a C17:0 fatty acid unit. If detected with LC/MS, |
| | applying electro-spray ionization (ESI) mass spectrometry, |
| | the mass-to-charge ratio (m/z) of the positively |
| | charged ionic species is 510.4 Da (+/−0.5 Da). |
| Lysophosphatidylcholine | Lysophosphatidylcholine (C18:0) represents the sum |
| (C18:0) | parameter of lysoglycerophosphorylcholines containing |
| | a C18:0 fatty acid unit. If detected with |
| | LC/MS, applying electro-spray ionization (ESI) mass |
| | spectrometry, the mass-to-charge ratio (m/z) of the |
| | positively charged ionic species is 546.6 Da (+/−0.5 Da). |
| Lysophosphatidylcholine | Lysophosphatidylcholine (C18:1) represents the sum |
| (C18:1) | parameter of lysoglycerophosphorylcholines |
| | containing a C18:1 fatty acid unit. If detected with |
| | LC/MS, applying electro-spray ionization (ESI) mass |
| | spectrometry, the mass-to-charge ratio (m/z) of the |
| | positively charged ionic species is 522.2 Da (+/−0.5 Da). |
| Lysophosphatidylcholine | Lysophosphatidylcholine (C18:2) represents the sum |
| (C18:2) | parameter of lysoglycerophosphorylcholines |
| | containing a C18:2 fatty acid unit. If detected with |
| | LC/MS, applying electro-spray ionization (ESI) mass |
| | spectrometry, the mass-to-charge ratio (m/z) of the |
| | positively charged ionic species is 542.4 Da (+/−0.5 Da). |
| Lysophosphatidylcholine | Lysophosphatidylcholine (C20:4) represents the sum |
| (C20:4) | parameter of lysoglycerophosphorylcholines |
| | containing a C20:4 fatty acid unit. If detected with |
| | LC/MS, applying eleclro-spray ionization (ESI) mass |
| | spectrometry, the mass-to-charge ratio (m/z) of the |
| | positively charged ionic species is 544.4 Da (+/−0.5 Da). |
| Lysophosphatidylethanolamine | Lysophosphatidylethanolamine (C22:5) exhibits the |
| (C22:5) | following characteristic ionic species when detected |
| | with LC/MS, applying electro-spray ionization (ESI) |
| | mass spectrometry: mass-to-charge ratio (m/z) of the |
| | positively charged ionic species is 528.2 (+/−0.5). |
| Phosphatidylcholine | Phosphatidylcholine (C16:0/C16:0) represents the |
| (C16:0, C16:0) | sum parameter of glycerophosphorylcholines |
| | containing either the combination of of two C16:0 fatty |
| | acid units. The mass-to-charge ratio (m/z) of the ionised |
| | species is 734.8 Da (+/−0.5 Da). |
| Phosphatidylcholine | Phosphatidylcholine (C16:0, C20:5) exhibits the following |
| (C16:0, C20:5) | characteristic ionic species when detected |
| | with LC/MS, applying electro-spray ionization (ESI) |
| | mass spectrometry: mass-to-charge ratio (m/z) of the |
| | positively charged ionic species is 780.8 (+/−0.5). |
| Phosphatidylcholine | Phosphatidylcholine (C16:1, C18:2) represents the |
| (C16:1, C18:2) | sum parameter of glycerophosphorylcholines |
| | containing the combination of a C16:1 fatty acid unit and |
| | a C18:2 fatty acid unit. If detected with LC/MS, applying |
| | electro-spray ionization (ESI) mass spectrometry, |
| | the mass-to-charge ratio (m/z) of the positively |
| | charged ionic species is 756.8 Da (+/−0.5 Da). |
| Phosphatidylcholine | Phosphatidylcholine (C18:0, C18:1) represents the |
| (C18:0, C18:1) | sum parameter of glycerophosphorylcholines |
| | containing the combination of a C18:0 fatty acid unit and |
| | a C18:1 fatty acid unit. If detected with LC/MS, applying |
| | electro-spray ionization (ESI) mass spectrometry, |
| | the mass-to-charge ratio (m/z) of the positively |
| | charged ionic species is 788.6 Da (+/−0.5 Da). |
| Phosphatidylcholine | Phosphatidylcholine (C18:0, C18:2) represents the |
| (C18:0, C18:2) | sum parameter of glycerophosphorylcholines |
| | containing the combination of a C18:0 fatty acid unit and |
| | a C18:2 fatty acid unit. If detected with LC/MS, applying |
| | electro-spray ionization (ESI) mass spectrometry, |
| | the mass-to-charge ratio (m/z) of the positively |
| | charged ionic species is 786.6 Da (+/−0.5 Da). |
| Phosphatidylcholine | Phosphatidylcholine (C18:0, C20:3) exhibits the following |
| (C18:0, C20:3) | characteristic ionic species when detected |
| | with LC/MS, applying electro-spray ionization (ESI) |
| | mass spectrometry: mass-to-charge ratio (m/z) of the |
| | positively charged ionic species is 812.6 (+/−0.5). |
| Phosphatidylcholine | Phosphatidylcholine (C18:0, C20:4) represents the |
| (C18:0, C20:4) | sum parameter of glycerophosphorylcholines |
| | containing the combination of a C18:0 fatty acid unit and |
| | a C20:4 fatty acid unit. If detected with LC/MS, applying |
| | electro-spray ionization (ESI) mass spectrometry, |
| | the mass-to-charge ratio (m/z) of the positively |
| | charged ionic species is 810.8 Da (+/−0.5 Da). |
| Phosphatidylcholine | Phosphatidylcholine (C18:0, C22:6) represents the |
| (C18:0, C22:6) | sum parameter of glycerophosphorylcholines |
| | containing the combination of a C18:0 fatty acid unit and |
| | a C22:6 fatty acid unit. If detected with LC/MS, applying |
| | electro-spray ionization (ESI) mass spectrometry, |
| | the mass-to-charge ratio (m/z) of the positively |
| | charged ionic species is 834.8 Da (+/−0.5 Da). |
| Phosphatidylcholine | Phosphatidylcholine (C16:0/C20:3 C18:1/C18:2) represents |
| (C18:1, C18:2) | the sum parameter of glycerophosphorylcholines |
| | containing the combination of a C18:1 fatty acid |
| | unit and a C18:2 fatty acid unit. The mass-to-charge |
| | ratio (m/z) of the ionised species is 784.6 Da (+/−0.5 Da). |
| Phosphatidylcholine | Phosphatidylcholine (C16:0/C22:6) represents the |
| (C16:0/C22:6) | sum parameter of glycerophosphorylcholines |
| | containing either the combination of a C16:0 fatty acid |
| | unit and a C22:6 fatty acid unit or the combination of |
| | a C18:2 fatty acid unit and a C20:4 fatty acid unit. |
| | The mass-to-charge ratio (m/z) of the ionised species |
| | is 806.6 Da (+/−0.5 Da). |
| Phosphatidylcholine | Metabolite belongs to the class of glycerophosphocholines. |
| No 02 | It exhibits the following characteristic ionic |
| | species when detected with LC/MS, applying electro- |
| | spray ionization (ESI) mass spectrometry: mass-to- |
| | charge ratio (m/z) of the positively charged ionic |
| | species is 808.4 (+/−0.5). |
| Phosphatidylcholine | Metabolite belongs to the class of glycerophosphocholines. |
| No 04 | It exhibits the following characteristic ionic |
| | species when detected with LC/MS, applying electro- |
| | spray ionization (ESI) mass spectrometry: mass-to- |
| | charge ratio (m/z) of the positively charged ionic |
| | species is 796.8 (+/−0.5). |
| Sphingomyelin (d18:1, C23:0) | Sphingomyelin (d18:1, C23:0) exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 801.8 (+/−0.5). |
| Sphingomyelin (d18:1, C24:0) | Sphingomyelin (d18:1, C24:0) represents the sum |
| | parameter of sphingomyelins containing the combination |
| | of a d18:1 long-chain base unit and a C24:0 |
| | fatty acid unit. If detected with LC/MS, applying electro- |
| | spray ionization (ESI) mass spectrometry, the |
| | mass-to-charge ratio (m/z) of the positively charged |
| | ionic species is 815.8 Da (+/−0.5 Da). |
| Sphingomyelin (d18:2, C16:0) | Sphingomyelin (d18:2, C16:0) exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 723.6 (+/−0.5). |
| Sphingomyelin (d18:2, C18:0) | Sphingomyelin (d18:2, C18:0) exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 729.8 (+/−0.5). |
| TAG (C16:0, C16:1) | Metabolite represents the sum of triacylglycerides |
| | containing the combination of a C16:0 fatty acid unit |
| | and a C16:1 fatty acid unit. It exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 549.6 (+/−0.5). |
| TAG (C16:0, C18:1, C18:3) | TAG (C16:0, C18:1, C18:3) exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 855.6 (+/−0.5). |
| TAG (C16:0, C18:2) | Metabolite represents the sum of triacylglycerides |
| | containing the combination of a C16:0 fatty acid unit |
| | and a C18:2 fatty acid unit. It exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 575.6 (+/−0.5). |
| TAG (C18:1, C18:2) | Metabolite represents the sum of triacylglycerides |
| | containing the combination of a C18:1 fatty acid unit |
| | and a C18:2 fatty acid unit. It exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 601.6 (+/−0.5). |
| TAG (C18:2, C18:2) | Metabolite represents the sum of triacylglycerides |
| | containing the combination of a C18:2 fatty acid unit |
| | and a C18:2 fatty acid unit. It exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 599.6 (+/−0.5). |
| TAG (C18:2, C18:3) | Metabolite represents the sum of triacylglycerides |
| | containing the combination of a C18:2 fatty acid unit |
| | and a C18:3 fatty acid unit. It exhibits the following |
| | characteristic ionic species when detected with |
| | LC/MS, applying electro-spray ionization (ESI) mass |
| | spectrometry: mass-to-charge ratio (m/z) of the positively |
| | charged ionic species is 597.6 (+/−0.5). |
| TAG (DAG-Fragment) | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 600.6 (+/−0.5). |
| TAG No 01 | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 547.6 (+/−0.5). |
| TAG No 02 | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 695.6 (+/−0.5). |
| TAG (C18:3, C18:2, C18:1) | TAG (C18:3, C18:2, C18:1) represents the sum parameter |
| | of triacylglycerides containing either the |
| | combination of a C18:3 fatty acid, a C18:2 fatty acid |
| | unit and a C18:1 fatty acid unit or the combination of |
| | a C20:4 fatty acid, a C18:2 fatty acid unit and a |
| | C16:0 fatty acid unit or the combination of a C20:5 |
| | fatty acid, a C18:1 fatty acid unit and a C16:0 fatty |
| | acid unit. Metabolite(s) belong to the class of triacyl- |
| | glycerides. It exhibits the following characteristic ionic |
| | species when detected with LC/MS, applying electro- |
| | spray ionization (ESI) mass spectrometry: mass- |
| | to-charge ratio (m/z) of the positively charged ionic |
| | species is 879.6 (+/−0.5). |
| TAG No 059 | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 904 (+/−0.5). |
| TAG No 07 | Metabolite belongs to the class of triacylglycerides. It |
| | exhibits the following characteristic ionic species |
| | when detected with LC/MS, applying electro-spray |
| | ionization (ESI) mass spectrometry: mass-to-charge |
| | ratio (m/z) of the positively charged ionic species is |
| | 853.6 (+/−0.5). |

TABLE 11
Compounds and dosage regimens

| | | | | Dosage | |
|---|---|---|---|---|---|
| Compound | Dose | Synonym | CAS no | administered | Details |
| 17-alpha- | high | 17-ethynyl- | 57-63-6 | 200 mg/kg by | in corn oil; |
| Ethynylestradiol | | 13-methyl- | | gavage | administration |
| | | 7,8,9,11,12, | | | volume: 5 |
| | | 13,14,15,16,17- | | | ml/kg body |
| | | decahydro- | | | weight |
| | | 6H-cyclo- | | | |
| | | penta[a]phen- | | | |
| | | anthrene-3,17- | | | |
| | | diol | | | |
| Apomorphine | high | 4H-Di- | 41372-20-7 | 0.25 mg/kg | in corn oil; |
| | | benzo(de,g)quino- | | body weight by | administration |
| | | line-10,11-diol | | gavage | volume: 5 |
| | | 5,6,6a,7- | | | ml/kg body |
| | | tetrahydro-6- | | | weight |
| | | methyl-(R)- | | | |
| | | (9CI) | | | |
| [3-(4,5-dihydro- | high | na | na | 8,000 ppm in | mixture in the |
| isoxazol-3-yl)-4- | | | | the diet | diet |
| methylsulfonyl- | | | | | |
| 2-chlor | | | | | |
| phenyl](5-hydroxy- | | | | | |
| 1-methyl-1H- | | | | | |
| pyrazol-4- | | | | | |
| yl)methanone | | | | | |
| Bromocriptine | high | na | 22260-51-1 | 10 mg/kg body | in aqua |
| Mesylate | | | | weight by | bidest. |
| | | | | gavage | containing 0.5% |
| | | | | | CMC (Tylose |
| | | | | | CB30000), |
| | | | | | administration |
| | | | | | volume: 10 |
| | | | | | ml/kg body |
| | | | | | weight |
| Cabergoline | high | Dostinex | 81409-90-7 | 5 mg/kg body | in drinking |
| | | | | weight by gavage | water containing |
| | | | | (days 0 to | 1% CMC; |
| | | | | 2), 2.5 mg/kg | administration |
| | | | | body weight by | volume: 10 |
| | | | | gavage (from | ml/kg bw. |
| | | | | day 3 on) | |
| Chlorpromazine | high | na | 50-53-3 | 10 mg/kg body | in 0.9% saline |
| | | | | weight intra- | solution, |
| | | | | peritoneal | administration |
| | | | | | volume: 1 |
| | | | | | ml/kg body |
| | | | | | weight |
| Citalopram | high | na | 59729-32-7 | 100 mg/kg body | in drinking |
| hydrobromide | | | | weight by | water containing |
| | | | | gavage | 0.5% CMC |
| | | | | | (Tylose |
| | | | | | CB30000), |
| | | | | | administration |
| | | | | | volume: 10 |
| | | | | | ml/kg bw |
| Dextroamphetamine | high | na | 51-63-8 | 10 mg/kg body | in 0.9% |
| sulfate | | | | weight sub- | saline; |
| | | | | cutanously | administration |
| | | | | | volume: 1 |
| | | | | | ml/kg bw |
| Escitalopram | high | na | 219861-08-2 | 100 mg/kg body | in drinking |
| oxalate | | | | weight by | water containing |
| | | | | gavage | 0.5% CMC |
| | | | | | (Tylose |
| | | | | | CB30000), |
| | | | | | administration |
| | | | | | volume: 10 |
| | | | | | ml/kg bw |
| Fluoxetine | high | na | 59333-67-4 | 20 mg/kg body | in drinking |
| hydrochloride | | | | weight by | water containing |
| | | | | gavage | 0.5% CMC |
| | | | | | (Tylose |
| | | | | | CB30000), |
| | | | | | administration |
| | | | | | volume: 10 |
| | | | | | ml/kg bw |
| NTBC (HPPD- | high | 2-(2-nitro-4- | na | 4 ppm in the | mixture in the |
| Inhibitor) | | trifluoro- | | diet | diet |
| | | methyl-benzoyl)- | | | |
| | | cyclohexane- | | | |
| | | 1,3-dione | | | |
| Olanzapine | high | Zyprexa | 132539-06-1 | 20 mg/kg body | in drinking |
| | | | | weight by | water containing |
| | | | | gavagel | 0.5% CMC |
| | | | | | (Tylose |
| | | | | | CB30000), |
| | | | | | administration |
| | | | | | volume: 10 |
| | | | | | ml/kg bw |
| Paroxetine | high | na | 61869-08-7 | 50 mg/kg body | in drinking |
| hydrochloride | | | | weight by | water containing |
| | | | | gavage | 0.5% CMC |
| | | | | | (Tylose |
| | | | | | CB30000), |
| | | | | | administration |
| | | | | | volume: 10 |
| | | | | | ml/kg bw |
| Pentobarbital | low | na | 57-33-0 | 12.5 mg/kg | in 0.9% NaCl; |
| sodium i.p. | | | | body i.p. | administration |
| | | | | | volume: 2 |
| | | | | | ml/kg bw |
| Pentoxifylline | high | na | 6493-05-6 | 600 mg/kg body | in drinking |
| | | | | weight by | water containing |
| | | | | gavage | 0.5% CMC |
| | | | | | (Tylose |
| | | | | | CB30000), |
| | | | | | administration |
| | | | | | volume: 10 |
| | | | | | ml/kg bw. |
| Phenobarbital | low | na | 57-30-7 | 100 ppm in the | mixture in the |
| sodium | | | | diet | diet |
| Phenytoin | low | Diphenylhydantoin | 57-41-0 | 600 ppm in the | mixture in the |
| | | | | diet | diet |
| Quetiapine | high | na | 111974-69-7 | 200 mg/kg body | in drinking |
| fumarate | | | | weight by | water containing |
| | | | | gavage | 0.5% CMC |
| | | | | | (Tylose |
| | | | | | CB30000), |
| | | | | | administration |
| | | | | | volume: 10 |
| | | | | | ml/kg bw |
| Raloxifene | high | Evista | 82640-04-8 | 600 mg/kg body | in drinking |
| Hydrochloride | | | | weight by | water containing |
| | | | | gavage | 0.5% CMC |
| | | | | | (Tylose |
| | | | | | CB30000), |
| | | | | | administration |
| | | | | | volume: 10 |
| | | | | | ml/kg bw |
| Risperidone | high | Risperdal | 106266-06-2 | 20 mg/kg body | in drinking |
| | | | | weight by | water containing |
| | | | | gavage | 0.5% CMC |
| | | | | | (Tylose |
| | | | | | CB30000), |
| | | | | | administration |
| | | | | | volume: 10 |
| | | | | | ml/kg bw |
| Selegiline | high | Movergan | 14611-52-0 | 50 mg/kg body | in drinking |
| hydrochloride | | | | weight by | water containing |
| | | | | gavage | 0.5% CMC |
| | | | | | (Tylose |
| | | | | | CB30000), |
| | | | | | administration |
| | | | | | volume: 10 |
| | | | | | ml/kg bw |
| Sertraline | high | na | 79617-96-2 | 100 mg/kg body | in drinking |
| hydrochloride | | | | weight by | water containing |
| | | | | gavage | 0.5% CMC |
| | | | | | (Tylose |
| | | | | | CB30000), |
| | | | | | administration |
| | | | | | volume: 10 |
| | | | | | ml/kg bw |
| Ziprasidone | high | na | 122883-93-6 | 150 mg/kg body | in drinking |
| hydrochloride | | | | weight by | water containing |
| | | | | gavage | 0.5% CMC |
| | | | | | (Tylose |
| | | | | | CB30000), |
| | | | | | administration |
| | | | | | volume: 10 |
| | | | | | ml/kg bw |

TABLE 12a
Markers for skeletal muscle innervation stimulation in male rats; Significant up-regulation changes (p-Value ≦0.1)
are marked in bold. All compounds were administered in high dose.

| | Toxa- | Toxa- | Toxa- | Glip- | Glip- | | Lead acetate | Lead acetate | Lead acetate | Thallium(I) | Thallium(I) | Thallium(I) |
|---|---|---|---|---|---|---|---|---|---|---|---|---|
| | phene | phene | phene | izide ( | izide | Glipizide | trihydrate | trihydrate ( | trihydrate ( | acetate | acetate | acetate |
| Compound | m7 | m14 | m28 | m7 | m14 | m28 | m7 | m14 | m28 | m7 | m14 | m28 |
| Creatinine | 1.41 | 1.45 | 1.37 | 1.00 | 1.99 | 1.33 | 1.16 | 1.32 | 1.66 | 1.69 | 1.50 | 1.89 |

TABLE 12b
Markers for skeletal muscle innervation stimulation in male rats; Significant down-regulation changes (p-Value ≦0.1)
are marked in bold. All compounds were administered in high dose.

| | | | | | | | Lead | Lead | | | | |
|---|---|---|---|---|---|---|---|---|---|---|---|---|
| | Toxa- | Toxa- | Toxa- | Glip- | Glip- | Glip- | acetate | acetate | Lead acetate | Thallium(I) | Thallium(I) | Thallium(I) |
| | phene | phene | phene | izide | izide | izide | trihydrate | trihydrate | trihydrate | acetate ( | acetate | acetate |
| Compound | m7 | m14 | m28 | m7 | m14 | m28 | m7 | m14 | m28 | m7 | m14 | m28 |
| Arginine | 0.96 | 0.78 | 0.77 | 0.79 | 0.93 | 0.88 | 0.86 | 0.87 | 0.90 | 0.97 | 0.87 | 0.84 |
| Citrulline | 0.87 | 1.07 | 1.10 | 0.92 | 0.85 | 0.96 | 0.94 | 0.90 | 0.91 | 0.84 | 0.84 | 0.94 |
| Tyrosine | 0.86 | 0.87 | 0.79 | 0.77 | 0.88 | 0.85 | 0.92 | 0.88 | 0.90 | 0.82 | 0.90 | 0.87 |
| Alanine | 0.82 | 0.87 | 0.83 | 0.62 | 0.68 | 0.89 | 0.68 | 0.75 | 0.75 | 0.81 | 0.95 | 0.79 |
| 5- | 0.98 | 0.87 | 0.81 | 0.74 | 0.81 | 0.78 | 0.88 | 0.92 | 0.86 | 0.92 | 0.87 | 0.83 |
| Oxoproline | | | | | | | | | | | | |
| Glutamine | 0.89 | 0.77 | 0.81 | 0.79 | 0.63 | 0.85 | 0.83 | 0.83 | 0.87 | 0.83 | 0.77 | 0.78 |
| Proline | 0.82 | 0.86 | 0.88 | 0.82 | 0.87 | 0.90 | 0.88 | 0.84 | 0.88 | 0.85 | 0.80 | 0.85 |
| Arginine | 0.87 | 0.79 | 0.84 | 0.80 | 0.86 | 0.88 | 0.91 | 0.90 | 0.89 | 0.90 | 0.80 | 0.85 |

1. A method for diagnosing neuronal toxicity comprising:
(a) determining the amount of at least one biomarker selected from any one of Tables 1a, 1b, 1c, 1d, 2a, 2b, 3a, 3b, 4a, 4b, 5a, 5b, 6a, 6b, 6c, 6d, 7a, 7b, 8a, 8b, 9a, 9b, 9c, 9d, 12a or 12b in a test sample of a subject suspected to suffer from neuronal toxicity, and
(b) comparing the amounts determined in step (a) to a reference, whereby neuronal toxicity is to be diagnosed.

2. The method of claim 1, wherein said subject has been brought into contact with a compound suspected to be capable of inducing neuronal toxicity.

3. A method of determining whether a compound is capable of inducing neuronal toxicity in a subject comprising:
(a) determining in a sample of a subject which has been brought into contact with a compound suspected to be capable of inducing neuronal toxicity the amount of at least one biomarker selected from any one of Tables 1a, 1b, 1c, 1d, 2a, 2b, 3a, 3b, 4a, 4b, 5a, 5b, 6a, 6b, 6c, 6d, 7a, 7b, 8a, 8b, 9a, 9b, 9c, 9d, 12a or 12b neuronal and
(b) comparing the amounts determined in step (a) to a reference, whereby the capability of the compound to induce neuronal toxicity is determined.

4. The method of claim 2, wherein said compound is at least one compound selected from the group consisting of: 17-alpha-Ethynylestradiol, Apomorphine, [3-(4,5-dihydro-isoxazol-3-yl)-4-methylsulfonyl-2-chlorphenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, and Thallium(I) acetate.

5. The method of claim 1, wherein said reference is derived from (i) a subject or group of subjects which suffers from neuronal toxicity or (ii) a subject or group of subjects which has been brought into contact with at least one compound selected from the group consisting of: 17-alpha-Ethynylestradiol, Apomorphine, [3-(4,5-dihydro-isoxazol-3-yl)-4-methylsulfonyl-2-chlorphenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, and Thallium(I) acetate.

6. The method of claim 5, wherein essentially identical amounts for the biomarkers in the test sample and the reference are indicative for neuronal toxicity.

7. The method of claim 1, wherein said reference is derived from (i) a subject or group of subjects known to not suffer from neuronal toxicity or (ii) a subject or group of subjects which has not been brought into contact with at least one compound selected from the group consisting of: 17-alpha-Ethynylestradiol, Apomorphine, [3-(4,5-dihydro-isoxazol-3-yl)-4-methylsulfonyl-2-chlorphenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, and Thallium(I) acetate.

8. The method of claim 1, wherein said reference is a calculated reference for the biomarkers for a population of subjects.

9. The method of claim 7, wherein amounts for the biomarkers which differ in the test sample in comparison to the reference are indicative for neuronal toxicity.

10. A method of identifying a substance for treating neuronal toxicity comprising the steps of:
(a) determining in a sample of a subject suffering from neuronal toxicity which has been brought into contact with a candidate substance suspected to be capable of treating neuronal toxicity the amount of at least one biomarker selected from any one of Tables 1a, 1b, 1c, 1d, 2a, 2b, 3a, 3b, 4a, 4b, 5a, 5b, 6a, 6b, 6c, 6d, 7a, 7b, 8a, 8b, 9a, 9b, 9c, 9d, 12a or 12b; and
(b) comparing the amounts determined in step (a) to a reference, whereby a substance capable of treating neuronal toxicity is to be identified.

11. The method of claim 10, wherein said reference is derived from (i) a subject or group of subjects which suffers from neuronal toxicity or (ii) a subject or group of subjects which has been brought into contact with at least one compound selected from the group consisting of: 17-alpha-Ethynylestradiol, Apomorphine, [3-(4,5-dihydro-isoxazol-3-yl)-4-methylsulfonyl-2-chlorphenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, and Thallium(I) acetate.

12. The method of claim 11, wherein amounts for the biomarkers which differ in the test sample and the reference are indicative for a substance capable of treating neuronal toxicity.

13. The method of claim 10, wherein said reference is derived from (i) a subject or group of subjects known to not suffer from neuronal toxicity or (ii) a subject or group of subjects which has not been brought into contact with at least one compound selected from the group consisting of: 17-alpha-Ethynylestradiol, Apomorphine, [3-(4,5-dihydro-isoxazol-3-yl)-4-methylsulfonyl-2-chlorphenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, and Thallium(I) acetate.

14. The method of claim 10, wherein said reference is a calculated reference for the biomarkers in a population of subjects.

15. The method of claim 13, wherein essentially identical amounts for the biomarkers in the test sample and the reference are indicative for a substance capable of treating neuronal toxicity.

16. (canceled)

17. A device for diagnosing neuronal toxicity in a sample of a subject suspected to suffer therefrom comprising:
(a) an analyzing unit comprising a detection agent for at least one biomarker selected from any one of Tables 1a, 1b, 1c, 1d, 2a, 2b, 3a, 3b, 4a, 4b, 5a, 5b, 6a, 6b, 6c, 6d, 7a, 7b, 8a, 8b, 9a, 9b, 9c, 9d, 12a or 12b which allows for determining the amount of the said biomarker present in the sample; and, operatively linked thereto,
(b) an evaluation unit comprising a stored reference and a data processor which allows for comparing the amount of the said at least one biomarker determined by the analyzing unit to the stored reference, whereby neuronal toxicity is diagnosed.

18. The device of claim 17, wherein said stored reference is a reference derived from a subject or a group of subjects known to suffer from neuronal toxicity or a subject or group of subjects which has been brought into contact with at least one compound selected from the group consisting of 17-alpha-Ethynylestradiol, Apomorphine, [3-(4,5-dihydro-isoxazol-3-yl)-4-methylsulfonyl-2-chlorphenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, and Thallium(I) acetate and said data processor executes instructions for comparing the amount of the at least one biomarker determined by the analyzing unit to the stored reference, wherein an essentially identical amount of the at least one biomarker in the test sample in comparison to the reference is indicative for the presence of neuronal toxicity or wherein an amount of the at least one biomarker in the test sample which differs in comparison to the reference is indicative for the absence of neuronal toxicity.

19. The device of claim 17, wherein said stored reference is a reference derived from a subject or a group of subjects known to not suffer from neuronal toxicity or a subject or group of subjects which has not been brought into contact with at least one compound selected from the group consisting of 17-alpha-Ethynylestradiol, Apomorphine, [3-(4,5-dihydro-isoxazol-3-yl)-4-methylsulfonyl-2-chlorphenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, and Thallium(I) acetate and said data processor executes instructions for comparing the amount of the at least one biomarker determined by the analyzing unit to the stored reference, wherein an amount of the at least one biomarker in the test sample which differs in comparison to the reference is indicative for the presence of neuronal toxicity or wherein an essentially identical amount of the at least one biomarker in the test sample in comparison to the reference is indicative for the absence of neuronal toxicity.

20. A kit for diagnosing neuronal toxicity comprising a detection agent for the at least one biomarker selected from any one of Tables 1a, 1b, 1c, 1d, 2a, 2b, 3a, 3b, 4a, 4b, 5a, 5b, 6a, 6b, 6c, 6d, 7a, 7b, 8a, 8b, 9a, 9b, 9c, 9d, 12a or 12b and standards for the at least one biomarker the concentration of which is derived from (i) a subject or a group of subjects known to suffer from neuronal toxicity or a subject or group of subjects which has been brought into contact with at least one compound selected from the group consisting of 17-alpha-Ethynylestradiol, Apomorphine, [3-(4,5-dihydro-isoxazol-3-yl)-4-methylsulfonyl-2-chlorphenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, Ziprasidone hydrochloride, Toxaphene, Glipizide, Lead acetate trihydrate, and Thallium(I) acetate or derived (ii) from a subject or a group of subjects known to not suffer from neuronal toxicity or a subject or group of subjects which has not been brought into contact with at least one compound selected from the group consisting of 17-alpha-Ethynylestradiol, Apomorphine, [3-(4,5-dihydro-isoxazol-3-yl)-4-methylsulfonyl-2-chlorphenyl](5-hydroxy-1-methyl-1H-pyrazol-4-yl)methanone, Bromocriptine Mesylate, Cabergoline, Chlorpromazine, Citalopram hydrobromide, Dextroamphetamine sulfate, Escitalopram oxalate, Fluoxetine hydrochloride, NTBC (HPPD-Inhibitor), Olanzapine, Paroxetine hydrochloride, Pentobarbital sodium i.p., Pentoxifylline, Phenobarbital sodium, Phenytoin, Quetiapine fumarate, Raloxifene Hydrochloride, Risperidone, Selegiline hydrochloride, Sertraline hydrochloride, and Ziprasidone hydrochloride, Toxaphene, Glipizide, Lead acetate trihydrate, and Thallium(I) acetate.
